(19) United States
(12) Patent Application Publication
(10) Pub. No.: US 2003/0153889 A1
(43) Pub. Date: Aug. 14, 2003

(54) Disposable absorbent garment with curved side seams

(76) Inventor: Bernadette Mary Gibbs, Statham, GA (US)

Correspondence Address: Christopher C. Campbell, Esq., Hunton & Williams, Suite 1200, 1900 K Street, NW, Washington, DC, 20006-1109, US

(21) Appl. No.: 10/074,028

(22) Filed: Feb. 14, 2002

(51) Int. Cl. A61F 13/15

(52) U.S. Cl. 604/385.3; 604/386

(57) ABSTRACT

An absorbent article is provided that comprises a main body having a first main body waist portion, a second main body waist portion and a central main body portion disposed between the first and second main body waist portions. The main body has orthogonal longitudinal and lateral axes. The absorbent article further comprises a pair of elastic side panels each connecting the first main body waist portion to the second main body waist portion. The main body and the side panels collectively define a waist opening edge surrounding a waist opening and two leg opening edges, each leg opening edge surrounding a leg opening. Each elastic side panel is formed from a first side portion that extends laterally outward from the first main body waist portion and terminates in a first outer lateral side panel edge. The side panel is also formed from a second side portion that extends laterally outward from the second main body waist portion and terminates in a second outer lateral side panel edge. The first side portion is attached to the second side portion by a side seam. At least a portion of the side seam is curved when the first and second side portions are in a fully stretched condition.

### BACKGROUND OF THE INVENTION

1. Field of the Invention

The invention relates generally to the field of absorbent garments, and more particularly to a disposable absorbent garment having a side seam, at least a portion of which is curved.

2. Description of Related Art

Disposable absorbent garments such as infant diapers or training pants, adult incontinence products, and other such products are well-known in the art. Typically, these garments comprise a main body formed from a liquid-permeable body-contacting liner sheet (or “topsheet”), a liquid-impermeable backing sheet (or “backsheet”) (collectively the “sheets”), and a moisture-absorbent core fiber (or “absorbent core”) that usually is made of a mat of randomly arrayed cellulose fiber and is generally disposed between the topsheet and the backsheet.

One type of disposable absorbent garment is the infant diaper, which is typically a flat, open-sided garment that is fitted about a child with the child laying down on top of the garment. The back portion of the garment is typically releasably connected to the front portion of the garment by a closure mechanism or fastener. Diapers are thus typically meant for use by children dependent upon a parent for assistance in putting on the absorbent garment.

Disposable diapers of this type are often replaced with pants-type diapers, or training pants, for older children who are able to partially dress themselves but still require absorbent undergarments. These pants-type diapers have closed sides and do not require a closure mechanism at the front or back of the garment. Because they are similar to conventional underwear, they can be put on and taken off with the child in a standing position. The construction of such garments is such that the garments can be easily donned by the child without assistance. This helps the child to make the transition between diapers and conventional underpants. Moreover, training pants are generally designed so that security against leakage can be maintained without parental adjustment.

Pant-like disposable absorbent garments are also available for adults. While the size and relative proportions of the components of these garments are different from the training pants garments, the overall construction is generally similar. In both types, the main body is doubled to provide front and back panels. The upper portions of the front and back panels are connected by stretchable side panels attached to the lateral edges of the main body. Leg holes are formed by the lower portion of the doubled body and the stretchable side panels. Typically, elastic leg gathers are provided to cause the garment to conform to the legs of the wearer thereby preventing leakage around the leg holes.

The configuration of the components of disposable absorbent garments are often driven in part by manufacturing methods wherein the garments are assembled in a continuously moving assembly line. The efficiency of these methods is dependent on the ability to continuously provide, place and attach the various components. This often requires that the designer of the garment consider such parameters as the speed and direction of motion of the garment during assembly and minimize the number of intermediate manufacturing steps that must be performed on the various components. These considerations have, at times, caused the sacrifice of features that could enhance the fit and comfort of the garments.

### SUMMARY OF THE INVENTION

The present invention provides an absorbent article comprising a main body having a first main body waist portion, a second main body waist portion and a central main body portion disposed between the first and second main body waist portions. The main body has orthogonal longitudinal and lateral axes. The absorbent article further comprises a pair of elastic side panels each connecting the first main body waist portion to the second main body waist portion. The main body and the side panels collectively define a waist opening edge surrounding a waist opening and two leg opening edges, each leg opening edge surrounding a leg opening. Each elastic side panel is formed from a first side portion that extends laterally outward from the first main body waist portion and terminates in a first outer lateral side panel edge. The side panel is also formed from a second side portion that extends laterally outward from the second main body waist portion and terminates in a second outer lateral side panel edge. The first side portion is attached to the second side portion by a side seam. At least a portion of the side seam is curved when the first and second side portions are in a fully stretched condition.

An aspect of the present invention provides an absorbent article comprising a backsheet. The main body of the absorbent article includes a first portion of the backsheet, a topsheet attached to the first portion of the backsheet and an absorbent core disposed intermediate the topsheet and the first portion of the backsheet. The side panels of the absorbent garment are formed at least in part from a second portion of the backsheet.

Another aspect of the present invention provides an absorbent article having side panels that are formed as separate members that are each attached to the main body along at least a portion of a main body lateral edge.

These and other objects, features and advantages of the invention will be apparent through the detailed description of the preferred embodiments and the drawings attached hereto. It also is to be understood that both the foregoing general description and the following detailed description are exemplary and explanatory and are not restrictive of the scope of the invention.

### BRIEF DESCRIPTION OF THE DRAWINGS

The invention may be understood more readily by viewing the drawings, in which:

FIG. 1 is a perspective view of a garment according to an embodiment of the invention as it would appear when worn by a user;

FIG. 2 is a partial cutaway view of an absorbent garment according to an embodiment of the invention, the garment being laid flat as it would appear during an intermediate stage of manufacture;

FIG. 3 is a front view of the garment of FIG. 2 upon completion of manufacture;

FIG. 4 is a partial cutaway view of an absorbent garment according to an embodiment of the invention, the garment being laid flat as it would appear during an intermediate stage of manufacture;

FIG. 5 is a front view of the garment of FIG. 4 upon completion of manufacture;

FIG. 6 is a partial cutaway view of an absorbent garment according to an embodiment of the invention, the garment being laid flat as it would appear during an intermediate stage of manufacture;

FIG. 7 is a front view of the garment of FIG. 6 upon completion of manufacture;

FIG. 8 is a partial cutaway view of an absorbent garment according to an embodiment of the invention, the garment being laid flat as it would appear during an intermediate stage of manufacture;

FIG. 9 is a front view of the garment of FIG. 8 upon completion of manufacture;

FIG. 10 is a partial cutaway view of an absorbent garment according to an embodiment of the invention, the garment being laid flat as it would appear during an intermediate stage of manufacture;

FIG. 11 is a front view of the garment of FIG. 10 upon completion of manufacture;

FIG. 12 is a partial cutaway view of an absorbent garment according to an embodiment of the invention, the garment being laid flat as it would appear during an intermediate stage of manufacture;

FIG. 13 is a front view of the garment of FIG. 12 upon completion of manufacture;

FIG. 14 is a perspective view of a garment according to an embodiment of the invention as it would appear when worn by a user;

FIG. 15 is a partial cutaway view of an absorbent garment according to an embodiment of the invention, the garment being laid flat as it would appear during an intermediate stage of manufacture;

FIG. 16 is a front view of the garment of FIG. 15 upon completion of manufacture;

FIG. 17 is a partial cutaway view of an absorbent garment according to an embodiment of the invention, the garment being laid flat as it would appear during an intermediate stage of manufacture;

FIG. 18 is a front view of the garment of FIG. 17 upon completion of manufacture;

FIG. 19 is a partial cutaway view of an absorbent garment according to an embodiment of the invention, the garment being laid flat as it would appear during an intermediate stage of manufacture;

FIG. 20 is a front view of the garment of FIG. 19 upon completion of manufacture;

FIG. 21 is a partial cutaway view of an absorbent garment according to an embodiment of the invention, the garment being laid flat as it would appear during an intermediate stage of manufacture;

FIG. 22 is a front view of the garment of FIG. 21 upon completion of manufacture;

FIG. 23 is a partial cutaway view of an absorbent garment according to an embodiment of the invention, the garment being laid flat as it would appear during an intermediate stage of manufacture;

FIG. 24 is a front view of the garment of FIG. 23 upon completion of manufacture;

FIG. 25 is a partial cutaway view of an absorbent garment according to an embodiment of the invention, the garment being laid flat as it would appear during an intermediate stage of manufacture; and

FIG. 26 is a front view of the garment of FIG. 25 upon completion of manufacture.

### DETAILED DESCRIPTION OF THE INVENTION

The present invention provides a pant-like absorbent garment with stretchable side panels that are curved so as to better conform to the body of the wearer, thereby providing a more comfortable fit. As used herein, the terms “absorbent garment” and “absorbent article” refer to garments that absorb and contain exudates, and more specifically, refer to garments that are placed against or in proximity to the body of the wearer to absorb and contain the various exudates discharged from the body. A non-exhaustive list of examples of absorbent garments includes diapers, diaper covers, disposable diapers, training pants, feminine hygiene products, and adult incontinence products. The term “disposable absorbent garment” refers to absorbent garments that are intended to be discarded or partially discarded after a single use (i.e., they are not intended to be laundered or otherwise restored or reused).

The present invention can be used with all of the foregoing classes of absorbent garments, without limitation, whether disposable or otherwise. These classifications are used interchangeably throughout the specification, but are not intended to limit the claimed invention. The invention will be understood to encompass, without limitation, all classes and types of absorbent garments, including those described above.

The term “component” can refer, but is not limited, to designated selected regions, such as edges, corners, sides or the like; structural members, such as elastic strips, absorbent pads, stretchable layers or panels, layers of material, or the like; or a graphic.

Absorbent garments and diapers may have a number of different constructions. In each of these constructions it is generally the case that an absorbent core is disposed between a liquid pervious, body-facing topsheet, and a liquid impervious, exterior backsheet. In some cases, one or both of the topsheet and backsheet may be shaped to form a pant-like garment. In other cases, the topsheet, backsheet and absorbent core may be formed as a discrete assembly that is placed on a main chassis layer and the chassis layer is shaped to form a pant-like garment. The garment may be provided to the consumer in the fully assembled pant-like shape, or may be partially pant-like and require the consumer to take the final steps necessary to form the final pant-like shape. In the case of training pant-type garments and most adult incontinent products, the garment is provided fully formed with factory-made side seams and the garment is donned by pulling it up the wearer's legs. In the case of diapers, a caregiver usually wraps the diaper around the wearer's waist and joins the side seams manually by attaching one or more adhesive or mechanical tabs, thereby forming a pant-like structure. For clarity, the present invention is described herein only with reference to a training pant-type garment in which the topsheet, backsheet and absorbent core are assembled into a main body that, when combined with side panels forms a pant-like garment, although the invention may be used with other constructions.

Throughout this description, the term “disposed” and the expressions “disposed on,” “disposing on,” “disposed in,” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “topsheet” and “backsheet” denote the relationship of these materials or layers with respect to an absorbent core. It is understood that additional layers may be present between the absorbent core and the topsheet and backsheet, and that additional layers and other materials may be present on the side opposite the absorbent core from either the topsheet or the backsheet.

The present invention provides an absorbent garment having a main body formed with a topsheet and a backsheet having an absorbent core disposed therebetween. The main body is a generally planar structure that is doubled to form a front main body waist portion and a rear main body waist portion connected by a crotch region. The front main body waist portion is connected to the rear main body waist portion by a pair of side panels. Each side panel may be formed from a generally planar side panel member that is attached to the main body along a lateral edge of the main body and is doubled along with the main body. Alternatively, the side panel members may be integrally formed with a backsheet of the main body. The side panel members may each have a relatively narrow central portion adjacent the crotch region of the main body and wider front and rear portions adjacent the front and rear main body waist portions, respectively. The front portion of each side panel member is joined to the rear portion of the side panel member adjacent the outer lateral edges of the front and rear portions to form a side panel seam. At least a portion of the side panel seam is curved so as to conform to the hip contour of a wearer. This seam configuration provides enhanced fit and comfort to the wearer. The front and rear main body waist portions and the side panels combine to define a waist opening and two leg openings.

Those skilled in the art will recognize that “front” and “rear” in the context of the invention denote for clarity purposes only the front and rear of a user, and that the absorbent garment could be reversed whereby the previously described “front” portion becomes the rear portion, and vice versa.

The invention will now be described with reference to the attached drawings, which illustrate various embodiments of the invention. For clarity, features that appear in more than one figure have the same reference number in each figure.

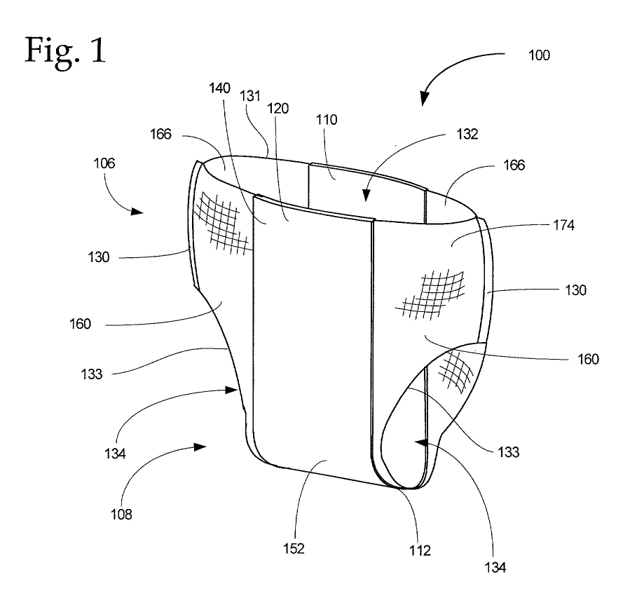

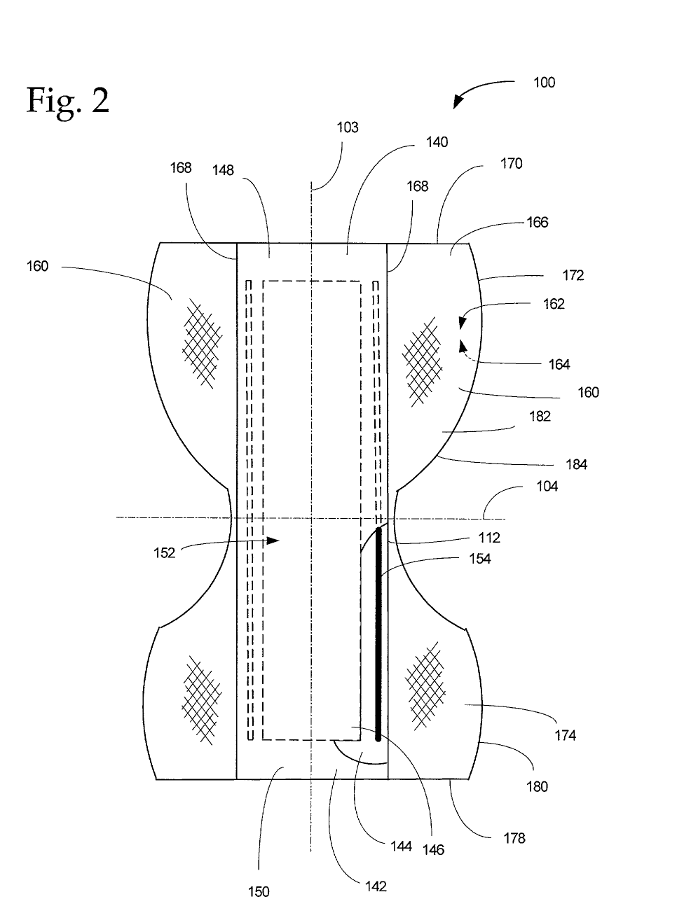

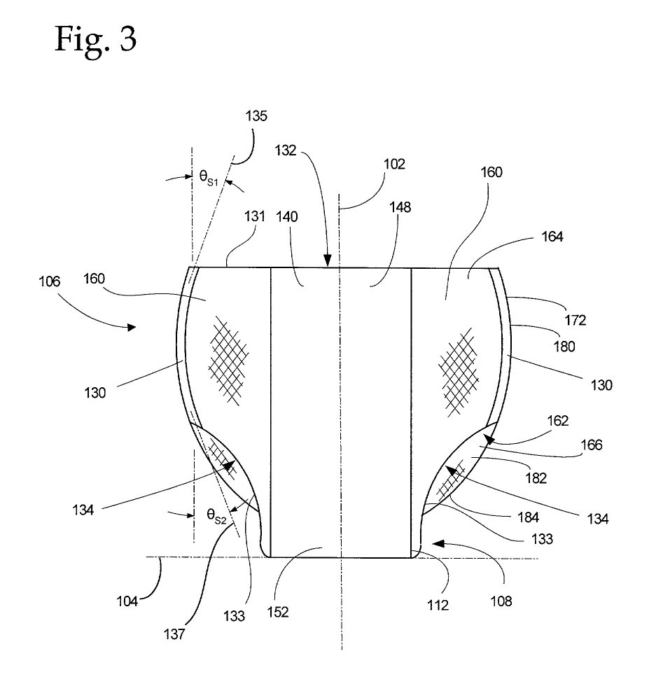

FIGS. 1-3 depict a preferred embodiment of an absorbent garment (preferably a disposable absorbent garment) 100 of the present invention. The absorbent garment 100 is depicted as and will be discussed as a child's training pant; however, this depiction is not intended to limit the invention, and those skilled in the art will appreciate that the invention covers other types of absorbent garments including incontinent pants for adults. The absorbent garment 100 is depicted in FIGS. 1 and 3 in its fully assembled form as it would appear when worn, with FIG. 1 being a perspective view and FIG. 3 a front view. FIG. 2 illustrates the garment in an intermediate stage of assembly. At this stage, the garment 100 is fully assembled except for the joinder at the side seams 130, which will be discussed in more detail hereafter. In FIG. 2, the garment 100 is shown in a generally flattened condition as it appears during the intermediate stage of assembly.

The garment 100 is shown with and will be described relative to first and second orthogonal axes 102, 104. The first axis 102 may be referred to as the vertical axis 102 and the second axis 104 may be referred to as the lateral axis 104. It will be understood by those having ordinary skill in the art that the vertical axis 102 corresponds generally to a head-to-toe axis of a standing wearer and that the lateral axis 104 corresponds generally to a side-to-side axis of a standing wearer.

Except where noted, in all drawings included herein, the elastic components of garments of the various embodiments of the invention are depicted in their relaxed condition with the effects of the elastics removed for clarity (when relaxed, the elastics typically cause the surrounding material to gather or “shirr”).

FIG. 2 is a plan view of the body-facing side of the flattened garment 100. In the flattened condition, the garment 100 may have a generally hourglass shaped structure, but it may also have any other shape suitable for the given application. The flattened absorbent garment 100 has a longitudinal axis 103 extending from the portion of the garment 100 that will conform to the front of the user to the portion of the garment that will conform to the rear of the user. The longitudinal axis 103 of the flattened garment 100 is orthogonal to the lateral axis 104, which, as previously noted, extends from side to side relative to a wearer of the finished garment 100.

In use, the garment 100 is a pant-like structure having a waist-encircling region 106 and a crotch region 108. The waist-encircling region 106 may include a first waist portion 110, disposable adjacent to, for example, the back waist area of a wearer's body, and a second waist portion 120, disposable adjacent to, for example, the front waist area of a wearer's body. The first and second waist portions 110, 120 may therefore correspond to the back and front of the wearer's body, respectively. The first and second waist portions 110, 120 are joined to one another at side seams 130 to form the waist encircling portion 106 and to define a waist opening edge 131 surrounding a waist opening 132 and leg opening edges 133 surrounding two leg openings 134.

An important advantage of the present invention is that at least a portion of each side seam 130 is curved so as to conform to the lower waist, hip or thigh contour of the wearer. Prior garments have had seams formed as a single straight seam or as a composite of straight line segments. Such seam geometries either do not conform to the wearer's body contour or require the seam and the materials of the garment to stretch to conform to the wearer's body contour. Further, garments having seams with multiple straight-line segments may have corners between segments that tend to show through the wearer's outer garments. As will be discussed in the context of the various embodiments, absorbent garments according to the present invention may have side seams that are curved along their entire length or side seams with specific portions curved to conform to specific body areas. The absorbent garments of the invention may also have one or more straight-line segments.

The absorbent garment 100 comprises a generally planar main body 140 folded or doubled to form a generally U-shaped structure. The main body 140 preferably comprises a topsheet 142, and a backsheet 144 that may be substantially coterminous with the topsheet 142. When the garment is worn, the topsheet 142 faces the wearer's body, and the backsheet 144 faces away from the wearer. An absorbent core 146 preferably is disposed between at least a portion of the topsheet 142 and the backsheet 144. The main body 140 has a first main body waist portion 148 configured to be disposable adjacent the back of the wearer's waist area and a second main body waist portion 150 configured to be disposable adjacent the front of the wearer's waist area. The main body 140 also has a central main body portion 152 that connects the first and second main body waist portions 148, 150 and forms at least a portion of the crotch region 108 when the main body 140 is doubled.

The topsheet 142 and backsheet 144 may be constructed from a wide variety of materials known in the art. The invention is not intended to be limited to any specific materials for these components. The topsheet 142 and backsheet can be shaped and sized according to the requirements of various types of absorbent garments, or to accommodate various user sizes. The topsheet 142 and backsheet 144 may, for example be configured to provide a main body 140 that is hourglass shaped, rectangular, trapezoidal, “T” shaped, or other geometry.

Embodiments of the present invention may further comprise various additional features. For example, one or more pairs of elastic gathers 154 may extend adjacent the lateral edges of the main body 140. The main body 140 may also comprise one or more waste containment systems, such as inboard standing leg gathers (not shown), which preferably extend from the second main body waist portion 150 to the first main body waist portion 148 along opposite sides of longitudinal center line 102. One or both of the first and second main body waist portions 148, 150 may also be equipped with strips of elastic waist foam or other elastically extensible material (not shown), which help contract the garment around the wearer's waist, providing improved fit and leakage prevention.

The absorbent garment 100 also includes side panels 160 extending laterally outward from the lateral edges 168 of the main body 140. Each side panel 160 may be formed using a single continuous piece of elastic material having a body facing surface 162 and an opposing outward facing surface 164 as shown in FIG. 3. Alternatively, the side panel 160 may be formed from multiple portions of elastic material. For example, one portion of material could be used in the area adjacent the first main body waist portion 148 and a second portion of material could be used in the area adjacent the second main body waist portion 150. It will be understood that if multiple portions of elastic material are used to form the side panel 160, the side panel 160 could be formed without elastic material adjacent the central main body portion 152.

The elastic material of the side panels 160 may include a single elastic or elasticized sheet or may be formed as an elasticized laminate.

Each side panel 160 preferably has a first side portion 166 attached to a lateral edge 168 of the main body 140 adjacent the first main body waist portion 148 and a portion of the central main body portion 152. The first side portion 166 has a waist opening side panel edge 170 and an outer lateral side panel edge 172. Each side panel 160 also has a second side portion 174 attached to the main body lateral edge 168 adjacent the second main body waist portion 150 and a portion of the central main body portion 152. The second side portion 174 also has a waist opening side panel edge 178 and an outer lateral side panel edge 180.

The first and second side portions 166, 174 are configured so that the body facing surface 162 in the area adjacent the outer lateral side panel edge 172 of the first side portion 166 may be made to engage the body facing surface 162 in the area adjacent the outer lateral side panel edge 180 of the second side portion 174 when the garment is folded to convert it from the configuration of FIG. 2 to the configuration of FIG. 3. With the body facing surface 162 of the first and second side portions 166, 174 so-engaged, the first and second side portions 166, 174 may be joined by the formation of the side seam 130 thereon. The first and second side seam portions 166, 174 are joined and the side seam 130 is formed with the first and second side seam portions 166, 174 in a fully stretched condition.

As used herein the term “seam” is used to indicate an area of contact between two surfaces wherein the contact is maintained by a mechanical, chemical, adhesive or other type of bond formed along a substantially continuous path. The seams of the present invention may be formed using any method known in the art or a combination of such methods. Examples of such methods include: adhesive bonding using hot melt adhesives, construction adhesives or both, chemical or solvent bonding, stitching, heat bonding, autogenous bonding, and ultrasonic welding. Each of these methods may be used to form a seam having a predetermined geometric or free-form path.

The first and second side portions 166, 174 may be configured so that at least a portion of the outer lateral side panel edge 172 of the first side portion 166 is a mirror image of at least a portion of the outer lateral side panel edge 180 of the second side portion 174. Further, the first and second side portions 166, 174 may be configured so that when the body facing surface 162 of the first and second side portions 166, 174 is engaged by folding the garment during manufacture, the waist opening side panel edges 170, 178 can be substantially aligned and at least a portion of the outer lateral side panel edges 172, 180 can be substantially aligned.

When the first and second side portions 166, 174 of both side panels 160 are joined, the first and second main body waist portions 148, 150 combine with the side panels 160 to form the waist encircling portion 106, the waist opening edge 131 and the waist opening 132. Also, the central main body portion 152 combines with the side panels 160 to form the crotch region 108, the leg opening edges 133 and the leg openings 134. The curved side seam 130 causes the completed side panels 160 to adopt a pre-formed curvature that is not provided by previous garments having straight seams or seams formed only from straight segments.

As shown in FIGS. 1-3, the side seam 130 may be curved along its entire length. While the illustrated curve of the side seam is a circular arc segment, it will be understood that the present invention is not limited to a particular curve geometry. The side seam 130 of embodiments of the present invention may use any curvature that serves to enhance comfort and fit by better conformance with body curves of the wearer. For example, the geometry of the side seam 130 may include other types of regular curve segments such as an ellipse or can be formed from a plurality of similar or disparate regular curve segments. The side seam 130 can also be formed at least in part with a substantially free-form curve geometry.

Each side seam 130 intersects the waist opening edge 131 and one of the leg opening edges 133. At the intersection with the waist opening edge 131, a tangent 135 to the centerline of the side seam 130 defines a first seam angle θ_{S1} relative to the vertical when the first and second side portions 166, 174 are in their fully stretched condition. At the intersection with the leg opening edge 133, a tangent 137 to the centerline of the side seam 130 defines a second seam angle θ_{S2} relative to the vertical when the first and second side portions 166, 174 are in their fully stretched condition. The first and second seam angles θ_{S1}, θ_{S2} may be designed specifically to accommodate different body types and sizes. For certain body types, the first and second seam angles θ_{S1}, θ_{S2} may be equal. For other body types, the first and second seam angles θ_{S1}, θ_{S2} may be significantly different. The first and second seam angles θ_{S1}, θ_{S2} may each practicably be in a range of about 0 degrees to about 80 degrees. To accommodate the most typical body shapes, the first and second seam angles θ_{S1}, θ_{S2} are each preferably in a range from about 10 degrees to about 50 degrees and most preferably in a range from about 25 degrees to about 35 degrees.

It will be understood that the final geometry of the waist opening side panel edges 170, 178 and the outer lateral side panel edges 172, 180 may be formed either before or after the side seam 130 is formed to join the first and second side portions 166, 174. In either case, the lateral side panel edges 172, 180 may be formed so as to substantially match the geometry of the side seam 130.

The first side portion 166 may include a lower hip region 182 that provides additional coverage of the back of the wearer in the area adjacent the wearer's hip region. As best seen in FIGS. 2 and 3, this area extends laterally outward and downward beyond the corresponding area of the second side portion 174. As shown, the lateral edge 184 of the lower hip region 182 is curved. For comfort and aesthetic reasons, the curvature of the lateral edge 184 may be a continuation of the curvature of the outer lateral side panel edge 172. It will be understood, however, that the lateral edge 184 need not have a continuous curve and, in fact, need not be curved at all.

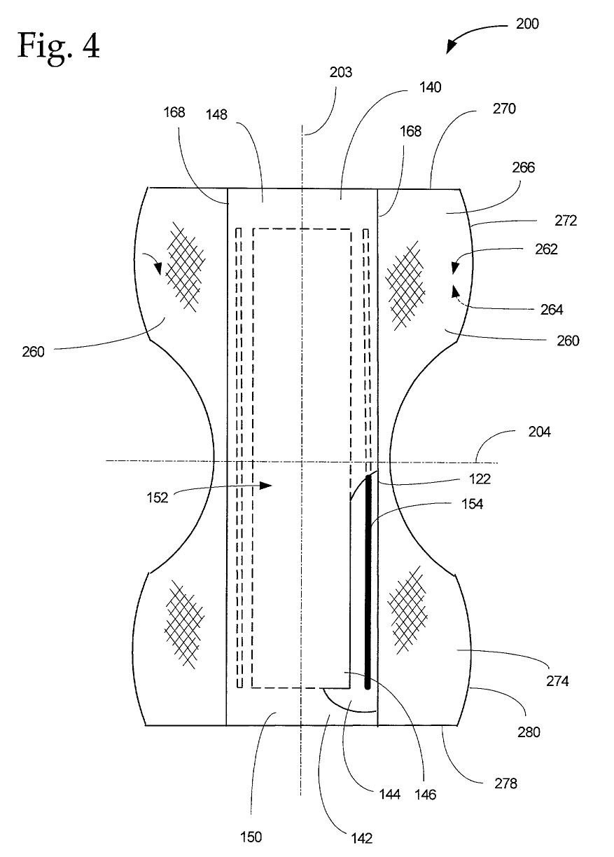

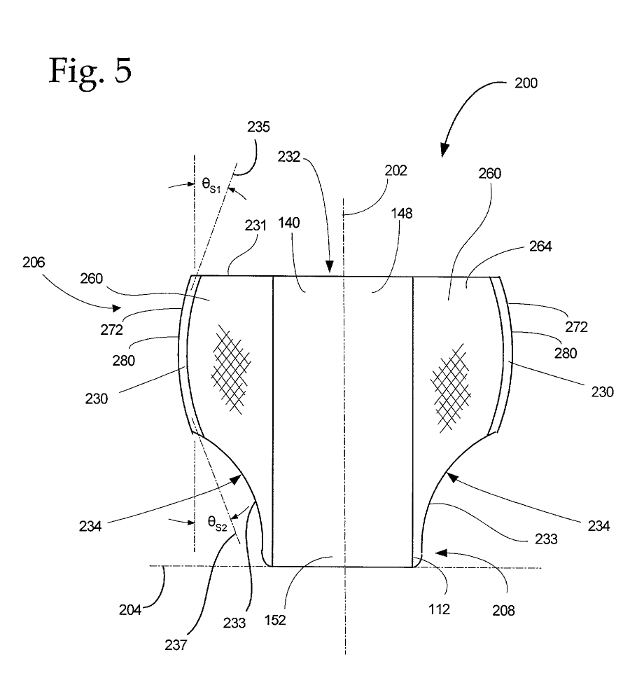

In an alternative embodiment of the invention illustrated in FIGS. 4 and 5, an absorbent garment 200 is provided that is similar to the absorbent garment of FIGS. 1-3. However, the first side portions 266 of the absorbent garment 200 do not include an extended lower hip portion. As shown in FIG. 4, this may result in a substantially symmetric hourglass-shaped structure prior to folding of the garment 200 and the joining of the first and second side portions 266, 274.

A front view of the absorbent garment 200 in its fully assembled form as it would appear when worn is shown in FIG. 5. FIG. 4 illustrates the garment 200 in an intermediate stage of assembly. At this stage, the garment 200 is fully assembled except for the joinder at the side seams 230. In FIG. 4, the garment 200 is shown in a generally flattened condition as it appears during the intermediate stage of assembly.

The garment 200 is shown with and will be described relative to first and second orthogonal axes 202, 204. The first axis 202 may be referred to as the vertical axis 202 and the second axis 204 may be referred to as the lateral axis 204. It will be understood by those having ordinary skill in the art that the vertical axis 202 corresponds generally to a head-to-toe axis of a standing wearer and that the lateral axis 204 corresponds generally to a side-to-side axis of a standing wearer.

FIG. 4 is a plan view of the body-facing side of the flattened garment 200. In the flattened condition, the garment 200 may have a generally symmetric hourglass shaped structure, but it may also have any other shape suitable for the given application. The flattened absorbent garment 200 has a longitudinal axis 203 extending from the portion of the garment 200 that will conform to the front of the user to the portion of the garment that will conform to the rear of the user. The longitudinal axis 203 of the flattened garment 200 is orthogonal to the lateral axis 204, which, as previously noted, extends from side to side relative to a wearer of the finished garment 200.

The absorbent garment 200 may have a main body 140 and associated components that are substantially similar to those of the absorbent garment 200 of FIGS. 1-3. The absorbent garment 200 includes side panels 260 extending laterally outward from the lateral edges 168 of the main body 140. Each side panel 260 may be formed using a single continuous piece of elastic material having a body facing surface 262 and an opposing outward facing surface 264 as shown in FIGS. 4 and 5. Alternatively, the side panel 260 may be formed from multiple portions of elastic material. For example, one portion of material could be used in the area adjacent the first main body waist portion 148 and a second portion of material could be used in the area adjacent the second main body waist portion 150. It will be understood that if multiple portions of elastic material are used to form the side panel 260, the side panel 260 could be formed without elastic material adjacent the central main body portion 152.

Each side panel 260 has a first side portion 266 attached to a lateral edge 168 of the main body 140 adjacent the first main body waist portion 148 and a portion of the central main body portion 152. The first side portion 266 has a waist opening side panel edge 270 and an outer lateral side panel edge 272. Each side panel 260 also has a second side portion 274 attached to the main body lateral edge 168 adjacent the second main body waist portion 150 and a portion of the central main body portion 152. The second side portion 274 also has a waist opening side panel edge 278 and an outer lateral side panel edge 280.

The first and second side portions 266, 274 are configured so that the body facing surface 262 in the area adjacent the outer lateral side panel edge 272 of the first side portion 266 may be made to engage the body facing surface 262 in the area adjacent the outer lateral side panel edge 280 of the second side portion 274 when the garment is folded to convert it from the configuration of FIG. 4 to the configuration of FIG. 5. With the body facing surface 262 of the first and second side portions 266, 274 so-engaged, the first and second side portions 266, 274 may be joined by the formation of the side seam 230 thereon. The first and second side seam portions 266, 274 are joined and the side seam 230 is formed with the first and second side seam portions 266, 274 in a fully stretched condition.

The first and second side portions 266, 274 may be configured so that at least a portion of the outer lateral side panel edge 272 of the first side portion 266 is a mirror image of at least a portion of the outer lateral side panel edge 280 of the second side portion 274. Further, the first and second side portions 266, 274 may be configured so that when the body facing surface 262 of the first and second side portions 266, 274 is engaged by folding the garment during manufacture, the waist opening side panel edges 270, 278 can be substantially aligned and at least a portion of the outer lateral side panel edges 272, 280 can be substantially aligned.

When the first and second side portions 266, 274 of both side panels 260 are joined, the first and second main body waist portions 148, 150 combine with the side panels 260 to form a waist encircling portion 206, a waist opening edge 231 and a waist opening 232. Also, the central main body portion 152 combines with the side panels 260 to form a crotch region 208, leg opening edges 233 and two leg openings 234. The curved side seam 230 causes the completed side panels 260 to adopt a pre-formed curvature that is not provided by previous garments having straight seams or seams formed only from straight segments.

The side seam 230 may be curved along its entire length. While the illustrated curve of the side seam is a circular arc segment, it will be understood that the present invention is not limited to a particular curve geometry. The side seam 230 of embodiments of the present invention may use any curvature that serves to enhance comfort and fit by better conformance with body curves of the wearer. For example, the geometry of the side seam 230 may include other types of regular curve segments such as an ellipse or can be formed from a plurality of similar or disparate regular curve segments. The side seam 230 can also be formed at least in part with a substantially free-form curve geometry.

Each side seam 230 intersects the waist opening edge 231 and one of the leg opening edges 233. At the intersection with the waist opening edge 231, a tangent 235 to the centerline of the side seam 230 defines a first seam angle θ_{S1} relative to the vertical. At the intersection with the leg opening edge 233, a tangent 236 to the centerline of the side seam 230 defines a second seam angle θ_{S2} relative to the vertical. The first and second seam angles θ_{S1}, θ_{S2} may be designed specifically to accommodate different body types and sizes. For certain body types, the first and second seam angles θ_{S1}, θ_{S2} may be equal. For other body types, the first and second seam angles θ_{S1}, θ_{S2} may be significantly different. The first and second seam angles θ_{S1}, θ_{S2} may each practicably be in a range of about 0 degrees to about 80 degrees. To accommodate the most typical body shapes, the first and second seam angles θ_{S1}, θ_{S2} are each preferably in a range from about 10 degrees to about 50 degrees and most preferably in a range from about 25 degrees to about 35 degrees.

It will be understood that the final geometry of the waist opening side panel edges 270, 278 and the outer lateral side panel edges 272, 280 may be formed either before or after the side seam 230 is formed to join the first and second side portions 266, 274. In either case, the lateral side panel edges 272, 280 may be formed so as to substantially match the geometry of the side seam 230.

For certain wearers, it may not be necessary to provide a garment with side seams that are curved over their entire length. It may, for example, be advantageous to provide a side seam that is substantially vertical over a portion of its length but then curves inward in the vicinity of the leg openings, the waist opening or both. The present invention therefore encompasses embodiments that have only a portion of the side seam curved.

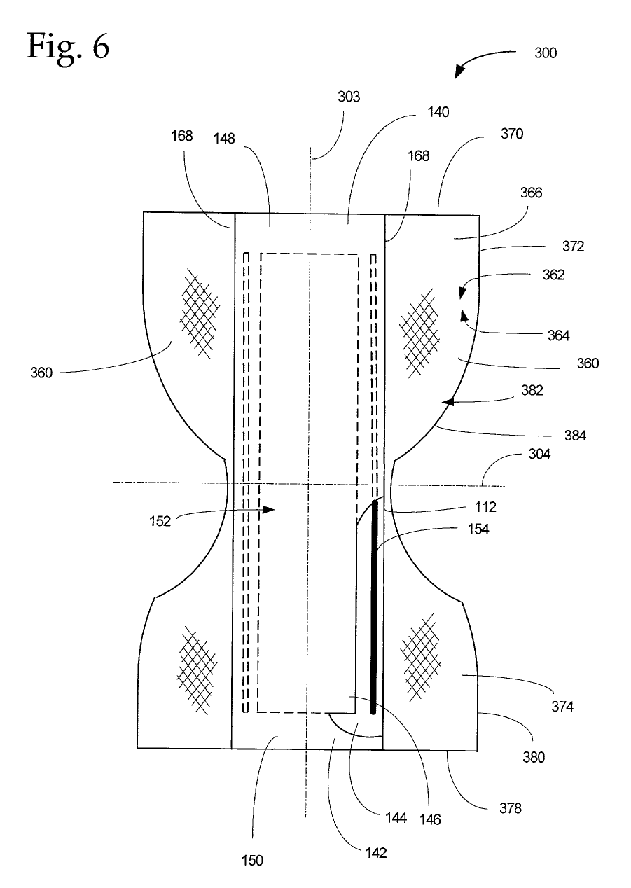

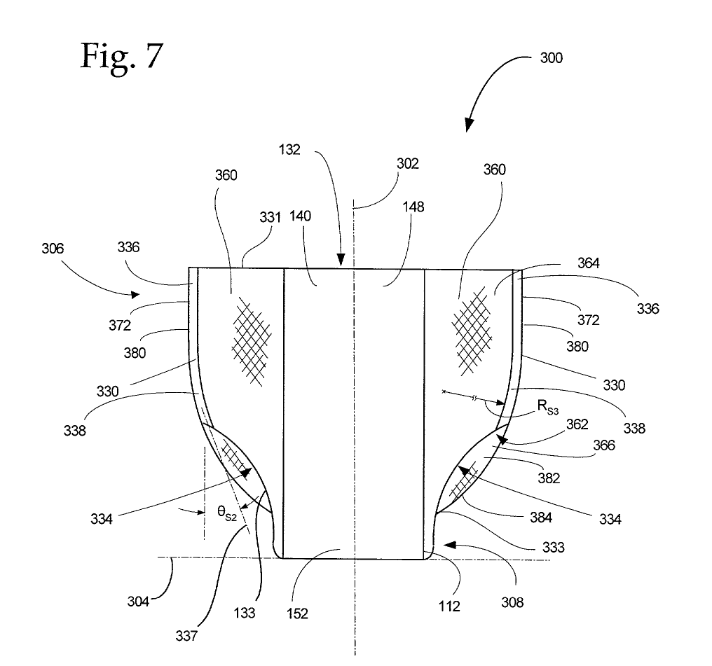

FIGS. 6 and 7 depict another exemplary embodiment of an absorbent garment 300 according to the invention. In the absorbent garment 300, the first and second side portions 366, 374 are joined by forming a side seam 330 that is substantially straight in the area adjacent the waist opening 332 and curved in the area adjacent the leg openings 334. This configuration may provide additional comfort and security to a wearer having, for example, a disproportionately small waist as compared to the wearer's hips and thighs.

A front view of the absorbent garment 300 in its fully assembled form as it would appear when worn is shown in FIG. 7. FIG. 6 illustrates the garment 300 in an intermediate stage of assembly. At this stage, the garment 300 is fully assembled except for the joinder at the side seams 330. In FIG. 6, the garment 300 is shown in a generally flattened condition as it appears during the intermediate stage of assembly.

The garment 300 is shown with and will be described relative to first and second orthogonal axes 302, 304. The first axis 302 may be referred to as the vertical axis 302 and the second axis 304 may be referred to as the lateral axis 304. It will be understood by those having ordinary skill in the art that the vertical axis 302 corresponds generally to a head-to-toe axis of a standing wearer and that the lateral axis 304 corresponds generally to a side-to-side axis of a standing wearer.

FIG. 6 is a plan view of the body-facing side of the flattened garment 300. In the flattened condition, the garment 300 may have a generally hourglass shaped structure, but it may also have any other shape suitable for the given application. The flattened absorbent garment 300 has a longitudinal axis 303 extending from the portion of the garment 300 that will conform to the front of the user to the portion of the garment that will conform to the rear of the user. The longitudinal axis 303 of the flattened garment 300 is orthogonal to the lateral axis 304, which, as previously noted, extends from side to side relative to a wearer of the finished garment 300.

The absorbent garment 300 may have a main body 140 and associated components that are substantially similar to those of the of the previously described embodiments. The absorbent garment 300 includes side panels 360 extending laterally outward from the lateral edges 168 of the main body 140. Each side panel 360 may be formed using a single continuous piece of elastic material having a body facing surface 362 and an opposing outward facing surface 364 as shown in FIGS. 6 and 7. Alternatively, the side panel 360 may be formed from multiple portions of elastic material. For example, one portion of material could be used in the area adjacent the first main body waist portion 148 and a second portion of material could be used in the area adjacent the second main body waist portion 150. It will be understood that if multiple portions of elastic material are used to form the side panel 360, the side panel 360 could be formed without elastic material adjacent the central main body portion 152.

Each side panel 360 has a first side portion 366 attached to a lateral edge 168 of the main body 140 adjacent the first main body waist portion 148 and a portion of the central main body portion 152. The first side portion 366 has a waist opening side panel edge 370 and an outer lateral side panel edge 372. Each side panel 360 also has a second side portion 374 attached to the main body lateral edge 168 adjacent the second main body waist portion 150 and a portion of the central main body portion 152. The second side portion 374 also has a waist opening side panel edge 378 and an outer lateral side panel edge 380.

The first and second side portions 366, 374 are configured so that the body facing surface 362 in the area adjacent the outer lateral side panel edge 372 of the first side portion 366 may be made to engage the body facing surface 362 in the area adjacent the outer lateral side panel edge 380 of the second side portion 374 when the garment is folded to convert it from the configuration of FIG. 6 to the configuration of FIG. 7. With the body facing surface 362 of the first and second side portions 366, 374 so-engaged, the first and second side portions 366, 374 may be joined by the formation of the side seam 330 thereon. The first and second side seam portions 366,374 are joined and the side seam 330 is formed with the first and second side seam portions 366, 374 in a fully stretched condition.

The first and second side portions 366, 374 may be configured so that at least a portion of the outer lateral side panel edge 372 of the first side portion 366 is a mirror image of at least a portion of the outer lateral side panel edge 380 of the second side portion 374. Further, the first and second side portions 366, 374 may be configured so that when the body facing surface 362 of the first and second side portions 366, 374 is engaged by folding the garment during manufacture, the waist opening side panel edges 370, 378 can be substantially aligned and at least a portion of the outer lateral side panel edges 372,380 can be substantially aligned.

When the first and second side portions 366, 374 of both side panels 360 are joined, the first and second main body waist portions 148, 150 combine with the side panels 360 to form a waist encircling portion 306, a waist opening edge 331 and a waist opening 332. Also, the central main body portion 152 combines with the side panels 360 to form a crotch region 308, leg opening edges 333 and two leg openings 334. The partially curved side seam 330 causes the completed side panels 360 to adopt a pre-formed curvature that is not provided by previous garments having straight seams or seams formed only from straight segments.

In this embodiment, each side seam 330 has a straight portion 336 adjacent the waist opening 332 and a curved portion 338 adjacent the leg opening 334 on the same lateral side of the garment 300 as the side seam 330. The curved portion 338 of side seam 330 may have any curvature that serves to enhance comfort and fit by better conformance with body curves of the wearer. For example, the geometry of the curved portion 338 may include regular curve segments such as circular or elliptical arcs or can be formed from a plurality of similar or disparate regular curve segments. The curved portion 338 may also be formed at least in part with a substantially free-form curve geometry.

Each side seam 330 intersects the waist opening edge 331 and one of the leg opening edges 333. The straight seam portion 336 defines a first seam angle with respect to the vertical at the intersection of the side seam 330 with the waist opening edge 331. In the embodiment illustrated in FIG. 7, the straight seam portion 336 is substantially vertical, thus making the first seam angle equal to 0 degrees. It will be understood, however, that in other embodiments, the straight seam portion 336 could be non-vertical, thus producing a non-zero first seam angle. At the intersection with the leg opening edge 333, a tangent to the centerline of the curved seam portion 338 defines a second seam angle θ_{S2} relative to the vertical. The first seam angle and the second seam angle θ_{S2} may be designed specifically to accommodate different body types and sizes. For certain body types, the first and second seam angles may be equal. For other body types, the first and second seam angles may be significantly different. The first and second seam angles may each practicably be in a range of about 0 degrees to about 80 degrees. To accommodate the most typical body shapes, the first and second seam angles are each preferably in a range from about 10 degrees to about 50 degrees and most preferably in a range from about 25 degrees to about 35 degrees.

It will be understood that the final geometry of the waist opening side panel edges 370, 378 and the outer lateral side panel edges 372, 380 may be formed either before or after the side seam 330 is formed to join the first and second side portions 366, 374. In either case, the lateral side panel edges 372, 380 may be formed so as to substantially match the geometry of the side seam 330.

The first side portions 366 of the absorbent garment 300 each include a lower hip region 382 that is similar to that described above for the absorbent garment of FIGS. 1-3. As shown in FIGS. 6 and 7, the lateral edge 384 of the lower hip region 382 is curved. For comfort and aesthetic reasons, the curvature of the lateral edge 384 may be a continuation of the curvature of the outer lateral side panel edge 372. It will be understood, however, that the lateral edge 384 need not have a continuous curve and, in fact, need not be curved at all.

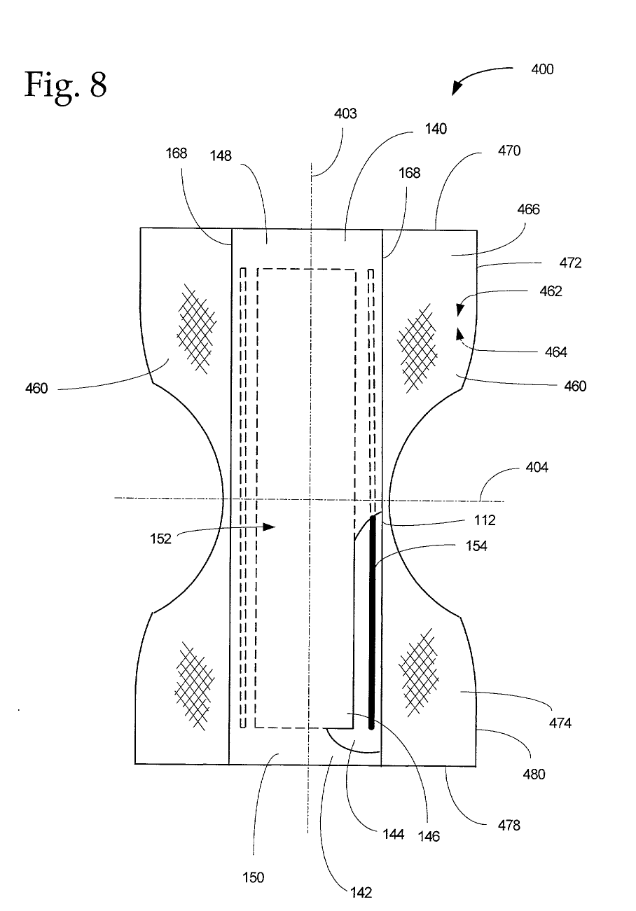

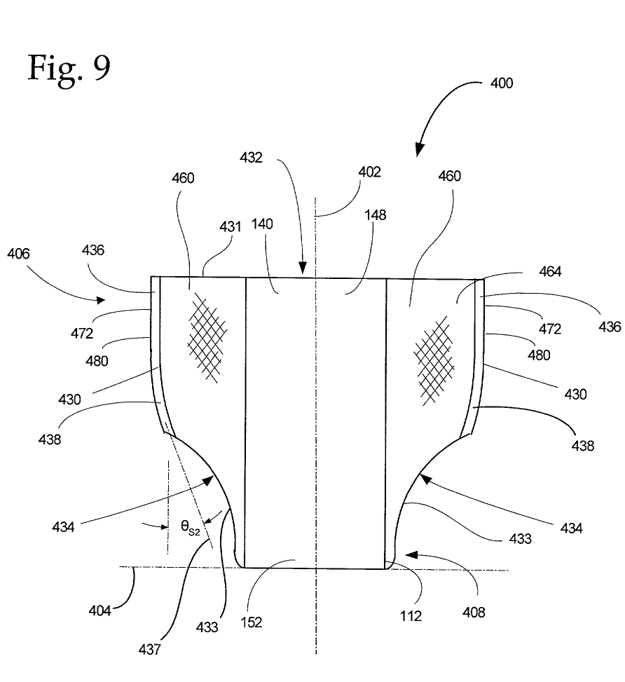

FIGS. 8 and 9 depict a similar absorbent garment 400 having first side portions 466 that do not have the lower hip region. A front view of the absorbent garment 400 in its fully assembled form as it would appear when worn is shown in FIG. 9. FIG. 8 illustrates the garment 400 in an intermediate stage of assembly. At this stage, the garment 400 is fully assembled except for the joinder at the side seams 430. In FIG. 8, the garment 400 is shown in a generally flattened condition as it appears during the intermediate stage of assembly.

The garment 400 is shown with and will be described relative to first and second orthogonal axes 402, 404. The first axis 402 may be referred to as the vertical axis 402 and the second axis 404 may be referred to as the lateral axis 404. It will be understood by those having ordinary skill in the art that the vertical axis 402 corresponds generally to a head-to-toe axis of a standing wearer and that the lateral axis 404 corresponds generally to a side-to-side axis of a standing wearer.

FIG. 8 is a plan view of the body-facing side of the flattened garment 400. In the flattened condition, the garment 400 may have a generally symmetric hourglass shaped structure, but it may also have any other shape suitable for the given application. The flattened absorbent garment 400 has a longitudinal axis 403 extending from the portion of the garment 400 that will conform to the front of the user to the portion of the garment that will conform to the rear of the user. The longitudinal axis 403 of the flattened garment 400 is orthogonal to the lateral axis 404, which, as previously noted, extends from side to side relative to a wearer of the finished garment 400.

The absorbent garment 400 may have a main body 140 and associated components that are substantially similar to those of the previously described embodiments. The absorbent garment 400 includes side panels 460 extending laterally outward from the lateral edges 168 of the main body 140. Each side panel 460 may be formed using a single continuous piece of elastic material having a body facing surface 462 and an opposing outward facing surface 464 as shown in FIGS. 8 and 9. Alternatively, the side panel 460 may be formed from multiple portions of elastic material. For example, one portion of material could be used in the area adjacent the first main body waist portion 148 and a second portion of material could be used in the area adjacent the second main body waist portion 150. It will be understood that if multiple portions of elastic material are used to form the side panel 460, the side panel 460 could be formed without elastic material adjacent the central main body portion 152.

Each side panel 460 has a first side portion 466 attached to a lateral edge 168 of the main body 140 adjacent the first main body waist portion 148 and a portion of the central main body portion 152. The first side portion 466 has a waist opening side panel edge 470 and an outer lateral side panel edge 472. Each side panel 460 also has a second side portion 474 attached to the main body lateral edge 168 adjacent the second main body waist portion 150 and a portion of the central main body portion 152. The second side portion 474 also has a waist opening side panel edge 478 and an outer lateral side panel edge 480.

The first and second side portions 466,474 are configured so that the body facing surface 462 in the area adjacent the outer lateral side panel edge 472 of the first side portion 466 may be made to engage the body facing surface 462 in the area adjacent the outer lateral side panel edge 480 of the second side portion 474 when the garment is folded to convert it from the configuration of FIG. 8 to the configuration of FIG. 9. With the body facing surface 462 of the first and second side portions 466, 474 so-engaged, the first and second side portions 466, 474 may be joined by the formation of the side seam 430 thereon. The first and second side seam portions 466, 474 are joined and the side seam 430 is formed with the first and second side seam portions 466,474 in a fully stretched condition.

The first and second side portions 466, 474 may be configured so that at least a portion of the outer lateral side panel edge 472 of the first side portion 466 is a mirror image of at least a portion of the outer lateral side panel edge 480 of the second side portion 474. Further, the first and second side portions 466, 474 may be configured so that when the body facing surface 462 of the first and second side portions 466, 474 is engaged by folding the garment during manufacture, the waist opening side panel edges 470, 478 can be substantially aligned and at least a portion of the outer lateral side panel edges 472, 480 can be substantially aligned.

When the first and second side portions 466, 474 of both side panels 460 are joined, the first and second main body waist portions 148, 150 combine with the side panels 460 to form a waist encircling portion 406, a waist opening edge 431 and a waist opening 432. Also, the central main body portion 152 combines with the side panels 460 to form a crotch region 408, leg opening edges 433 and two leg openings 434. The partially curved side seam 430 causes the completed side panels 460 to adopt a pre-formed curvature that is not provided by previous garments having straight seams or seams formed only from straight segments.

In this embodiment, each side seam 430 has a straight portion 436 adjacent the waist opening 432 and a curved portion 438 adjacent the leg opening 434 on the same lateral side of the garment 400 as the side seam 430. The curved portion 438 of side seam 430 may have any curvature that serves to enhance comfort and fit by better conformance with body curves of the wearer. For example, the geometry of the curved portion 438 may include regular curve segments such as circular or elliptical arcs or can be formed from a plurality of similar or disparate regular curve segments. The curved portion 438 may also be formed at least in part with a substantially free-form curve geometry.

Each side seam 430 intersects the waist opening edge 431 and one of the leg opening edges 433. The straight seam portion 436 defines a first seam angle with respect to the vertical at the intersection of the side seam 430 with the waist opening edge 431. In the embodiment illustrated in FIG. 9, the straight seam portion 436 is substantially vertical, thus making the first seam angle equal to 0 degrees. It will be understood, however, that in other embodiments, the straight seam portion 436 could be non-vertical, thus producing a non-zero first seam angle. At the intersection with the leg opening edge 433, a tangent to the centerline of the curved seam portion 438 defines a second seam angle θ_{S2} relative to the vertical. The first seam angle and the second seam angle θ_{S2} may be designed specifically to accommodate different body types and sizes. For certain body types, the first and second seam angles may be equal. For other body types, the first and second seam angles may be significantly different. The first and second seam angles may each practicably be in a range of about 0 degrees to about 80 degrees. To accommodate the most typical body shapes, the first and second seam angles are each preferably in a range from about 10 degrees to about 50 degrees and most preferably in a range from about 25 degrees to about 35 degrees.

It will be understood that the final geometry of the waist opening side panel edges 470, 478 and the outer lateral side panel edges 472, 480 may be formed either before or after the side seam 430 is formed to join the first and second side portions 466, 474. In either case, the lateral side panel edges 472, 480 may be formed so as to substantially match the geometry of the side seam 430.

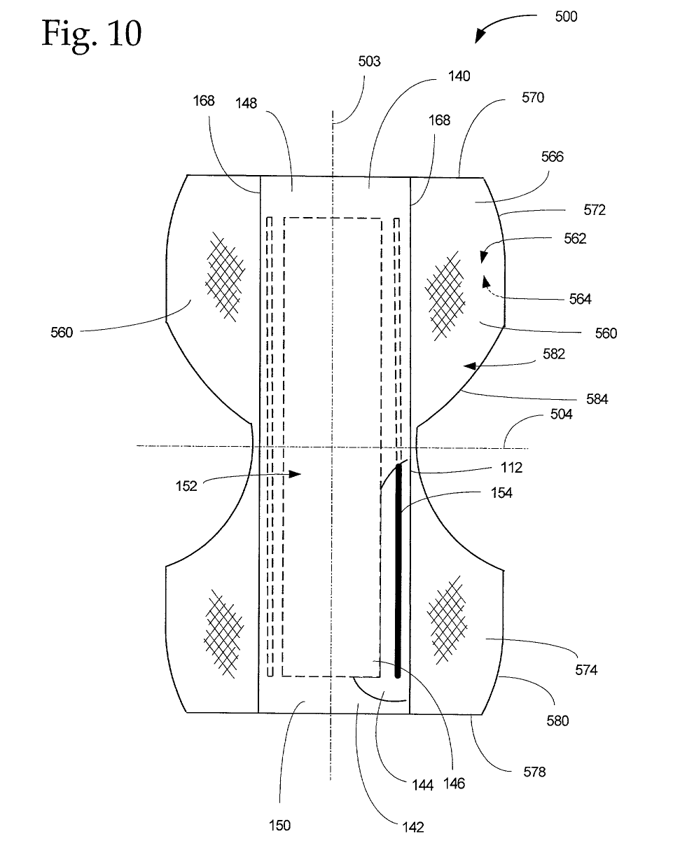

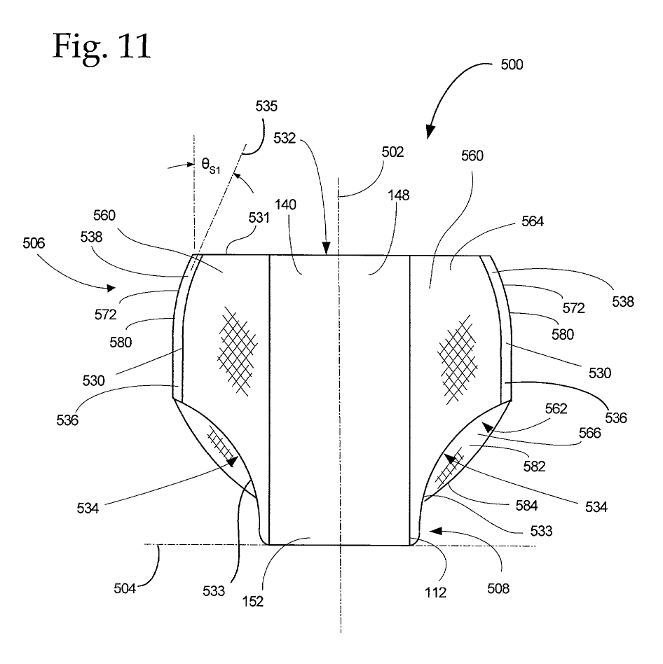

In another exemplary embodiment depicted in FIGS. 10 and 11, an absorbent garment 500 has first and second side portions 566, 574 that are joined by forming a side seam 530 that is substantially straight in the areas adjacent the leg openings 534 and curved in the area adjacent the waist opening 532. This configuration may provide additional comfort and leakage security in wearers having disproportionately small thighs as compared to the wearer's waist. The first side portions 566 of the garment 500 each include a lower hip region 582 that is similar to that described above for the absorbent garment of FIGS. 1-3.

A front view of the absorbent garment 500 in its fully assembled form as it would appear when worn is shown in FIG. 11. FIG. 10 illustrates the garment 500 in an intermediate stage of assembly. At this stage, the garment 500 is fully assembled except for the joinder at the side seams 530. In FIG. 10, the garment 500 is shown in a generally flattened condition as it appears during the intermediate stage of assembly.

The garment 500 is shown with and will be described relative to first and second orthogonal axes 502, 504. The first axis 502 may be referred to as the vertical axis 502 and the second axis 504 may be referred to as the lateral axis 504. It will be understood by those having ordinary skill in the art that the vertical axis 502 corresponds generally to a head-to-toe axis of a standing wearer and that the lateral axis 504 corresponds generally to a side-to-side axis of a standing wearer.

FIG. 10 is a plan view of the body-facing side of the flattened garment 500. In the flattened condition, the garment 500 may have a generally hourglass shaped structure, but it may also have any other shape suitable for the given application. The flattened absorbent garment 500 has a longitudinal axis 503 extending from the portion of the garment 500 that will conform to the front of the user to the portion of the garment that will conform to the rear of the user. The longitudinal axis 503 of the flattened garment 500 is orthogonal to the lateral axis 504, which, as previously noted, extends from side to side relative to a wearer of the finished garment 500.

The absorbent garment 500 may have a main body 140 and associated components that are substantially similar to those of the of the previously described embodiments. The absorbent garment 500 includes side panels 560 extending laterally outward from the lateral edges 168 of the main body 140. Each side panel 560 may be formed using a single continuous piece of elastic material having a body facing surface 562 and an opposing outward facing surface 564 as shown in FIGS. 10 and 11. Alternatively, the side panel 560 may be formed from multiple portions of elastic material. For example, one portion of material could be used in the area adjacent the first main body waist portion 148 and a second portion of material could be used in the area adjacent the second main body waist portion 150. It will be understood that if multiple portions of elastic material are used to form the side panel 560, the side panel 560 could be formed without elastic material adjacent the central main body portion 152.

Each side panel 560 has a first side portion 566 attached to a lateral edge 168 of the main body 140 adjacent the first main body waist portion 148 and a portion of the central main body portion 152. The first side portion 566 has a waist opening side panel edge 570 and an outer lateral side panel edge 572. Each side panel 560 also has a second side portion 574 attached to the main body lateral edge 168 adjacent the second main body waist portion 150 and a portion of the central main body portion 152. The second side portion 574 also has a waist opening side panel edge 578 and an outer lateral side panel edge 580.

The first and second side portions 566, 574 are configured so that the body facing surface 562 in the area adjacent the outer lateral side panel edge 572 of the first side portion 566 may be made to engage the body facing surface 562 in the area adjacent the outer lateral side panel edge 580 of the second side portion 574 when the garment is folded to convert it from the configuration of FIG. 10 to the configuration of FIG. 11. With the body facing surface 562 of the first and second side portions 566, 574 so-engaged, the first and second side portions 566, 574 may be joined by the formation of the side seam 530 thereon. The first and second side seam portions 566, 574 are joined and the side seam 530 is formed with the first and second side seam portions 566, 574 in a fully stretched condition.

The first and second side portions 566, 574 may be configured so that at least a portion of the outer lateral side panel edge 572 of the first side portion 566 is a mirror image of at least a portion of the outer lateral side panel edge 580 of the second side portion 574. Further, the first and second side portions 566, 574 may be configured so that when the body facing surface 562 of the first and second side portions 566, 574 is engaged by folding the garment during manufacture, the waist opening side panel edges 570, 578 can be substantially aligned and at least a portion of the outer lateral side panel edges 572, 580 can be substantially aligned.

When the first and second side portions 566, 574 of both side panels 560 are joined, the first and second main body waist portions 148, 150 combine with the side panels 560 to form a waist encircling portion 506, a waist opening edge 531 and a waist opening 532. Also, the central main body portion 152 combines with the side panels 560 to form a crotch region 508, leg opening edges 533 and two leg openings 534. The partially curved side seam 530 causes the completed side panels 560 to adopt a pre-formed curvature that is not provided by previous garments having straight seams or seams formed only from straight segments.

In this embodiment, each side seam 530 has a straight portion 536 adjacent the leg opening 534 on the same lateral side of the garment 500 as the side seam 530 and a curved portion 538 adjacent the waist opening 532. The curved portion 538 of side seam 530 may have any curvature that serves to enhance comfort and fit by better conformance with body curves of the wearer. For example, the geometry of the curved portion 538 may include regular curve segments such as circular or elliptical arcs or can be formed from a plurality of similar or disparate regular curve segments. The curved portion 538 may also be formed at least in part with a substantially free-form curve geometry.

Each side seam 530 intersects the waist opening edge 531 and one of the leg opening edges 533. At the intersection with the waist opening edge 531, a tangent to the centerline of the side seam 530 defines a first seam angle θ_{S1} relative to the vertical. The straight seam portion 536 defines a second seam angle with respect to the vertical at the intersection of the side seam 530 with the leg opening edge 533. In the embodiment illustrated in FIG. 11, the straight seam portion 536 is substantially vertical, thus making the second seam angle equal to 0 degrees. It will be understood, however, that in other embodiments, the straight seam portion 536 could be non-vertical, thus producing a non-zero second seam angle. The first seam angle θ_{S1} and the second seam angle may be designed specifically to accommodate different body types and sizes. For certain body types, the first and second seam angles may be equal. For other body types, the first and second seam angles may be significantly different. The first and second seam angles may each practicably be in a range of about 0 degrees to about 80 degrees. To accommodate the most typical body shapes, the first and second seam angles are each preferably in a range from about 10 degrees to about 50 degrees and most preferably in a range from about 25 degrees to about 35 degrees.

It will be understood that the final geometry of the waist opening side panel edges 570, 578 and the outer lateral side panel edges 572, 580 may be formed either before or after the side seam 530 is formed to join the first and second side portions 566, 574. In either case, the lateral side panel edges 572, 580 may be formed so as to substantially match the geometry of the side seam 530.

The first side portion 566 includes a lower hip region 582 that provides additional coverage of the back of the wearer in the area adjacent the wearer's hip region. This area extends laterally outward and downward beyond the corresponding area of the second side portion 574. Although the lateral edge 584 of the lower hip region 182 is shown as curved, it will be understood that the lateral edge 584 may be straight or a combination of curved and straight.

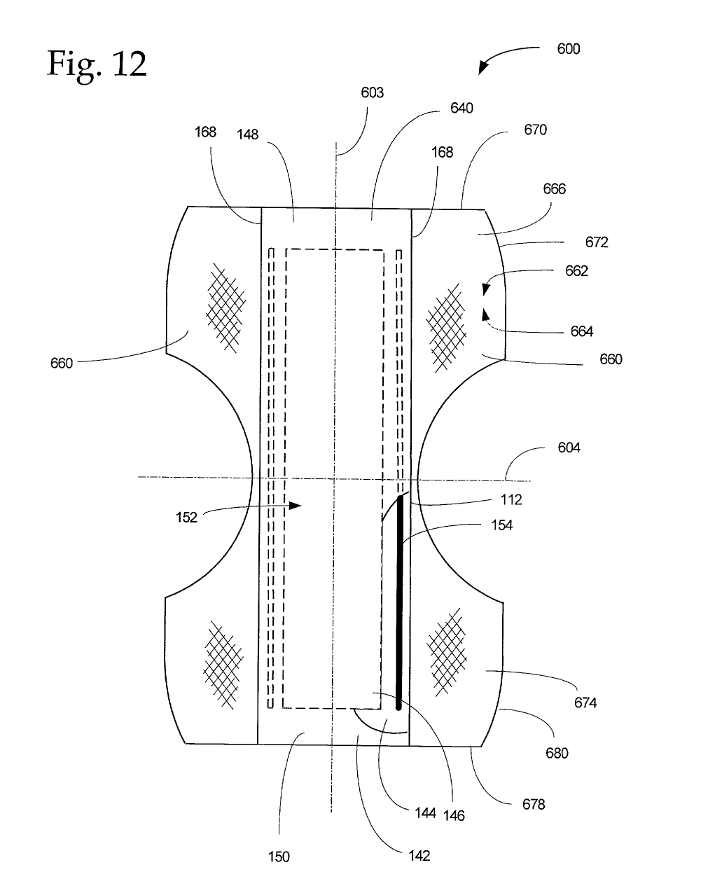

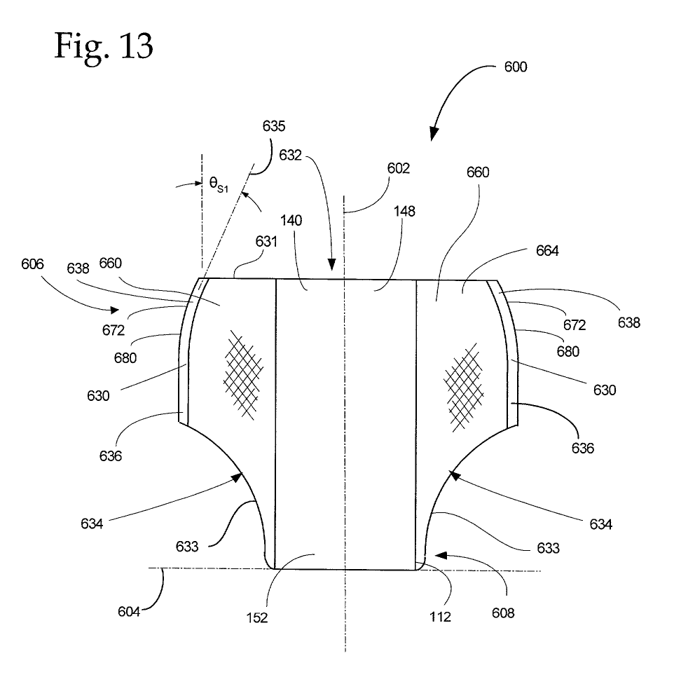

FIGS. 12 and 13 depict a similar absorbent garment 600 with first side portions 666 that do not have the lower hip region. A front view of the absorbent garment 600 in its fully assembled form as it would appear when worn is shown in FIG. 13. FIG. 12 illustrates the garment 600 in an intermediate stage of assembly. At this stage, the garment 600 is fully assembled except for the joinder at the side seams 630. In FIG. 12, the garment 600 is shown in a generally flattened condition as it appears during the intermediate stage of assembly.

The garment 600 is shown with and will be described relative to first and second orthogonal axes 602, 604. The first axis 602 may be referred to as the vertical axis 602 and the second axis 604 may be referred to as the lateral axis 604. It will be understood by those having ordinary skill in the art that the vertical axis 602 corresponds generally to a head-to-toe axis of a standing wearer and that the lateral axis 604 corresponds generally to a side-to-side axis of a standing wearer.

FIG. 12 is a plan view of the body-facing side of the flattened garment 600. In the flattened condition, the garment 600 may have a generally symmetric hourglass shaped structure, but it may also have any other shape suitable for the given application. The flattened absorbent garment 600 has a longitudinal axis 603 extending from the portion of the garment 600 that will conform to the front of the user to the portion of the garment that will conform to the rear of the user. The longitudinal axis 603 of the flattened garment 600 is orthogonal to the lateral axis 604, which, as previously noted, extends from side to side relative to a wearer of the finished garment 600.

The absorbent garment 600 may have a main body 140 and associated components that are substantially similar to those of the of the previously described embodiments. The absorbent garment 600 includes side panels 660 extending laterally outward from the lateral edges 168 of the main body 140. Each side panel 660 may be formed using a single continuous piece of elastic material having a body facing surface 662 and an opposing outward facing surface 664 as shown in FIGS. 12 and 13. Alternatively, the side panel 660 may be formed from multiple portions of elastic material. For example, one portion of material could be used in the area adjacent the first main body waist portion 148 and a second portion of material could be used in the area adjacent the second main body waist portion 150. It will be understood that if multiple portions of elastic material are used to form the side panel 660, the side panel 660 could be formed without elastic material adjacent the central main body portion 152.

Each side panel 660 has a first side portion 666 attached to a lateral edge 168 of the main body 140 adjacent the first main body waist portion 148 and a portion of the central main body portion 152. The first side portion 666 has a waist opening side panel edge 670 and an outer lateral side panel edge 672. Each side panel 660 also has a second side portion 674 attached to the main body lateral edge 168 adjacent the second main body waist portion 150 and a portion of the central main body portion 152. The second side portion 674 also has a waist opening side panel edge 678 and an outer lateral side panel edge 680.

The first and second side portions 666, 674 are configured so that the body facing surface 662 in the area adjacent the outer lateral side panel edge 672 of the first side portion 666 may be made to engage the body facing surface 662 in the area adjacent the outer lateral side panel edge 680 of the second side portion 674 when the garment is folded to convert it from the configuration of FIG. 12 to the configuration of FIG. 13. With the body facing surface 662 of the first and second side portions 666, 674 so-engaged, the first and second side portions 666, 674 may be joined by the formation of the side seam 630 thereon. The first and second side seam portions 666, 674 are joined and the side seam 630 is formed with the first and second side seam portions 666, 674 in a fully stretched condition.

The first and second side portions 666, 674 may be configured so that at least a portion of the outer lateral side panel edge 672 of the first side portion 666 is a mirror image of at least a portion of the outer lateral side panel edge 680 of the second side portion 674. Further, the first and second side portions 666, 674 may be configured so that when the body facing surface 662 of the first and second side portions 666, 674 is engaged by folding the garment during manufacture, the waist opening side panel edges 670, 678 can be substantially aligned and at least a portion of the outer lateral side panel edges 672, 680 can be substantially aligned.

When the first and second side portions 666, 674 of both side panels 660 are joined, the first and second main body waist portions 148, 150 combine with the side panels 660 to form a waist encircling portion 606, a waist opening edge 631 and a waist opening 632. Also, the central main body portion 152 combines with the side panels 660 to form a crotch region 608, leg opening edges 633 and two leg openings 634. The partially curved side seam 630 causes the completed side panels 660 to adopt a pre-formed curvature that is not provided by previous garments having straight seams or seams formed only from straight segments.

In this embodiment, each side seam 630 has a straight portion 636 adjacent the leg opening 634 on the same lateral side of the garment 600 as the side seam 630 and a curved portion 638 adjacent the waist opening 632. The curved portion 638 of side seam 630 may have any curvature that serves to enhance comfort and fit by better conformance with body curves of the wearer. For example, the geometry of the curved portion 638 may include regular curve segments such as circular or elliptical arcs or can be formed from a plurality of similar or disparate regular curve segments. The curved portion 638 may also be formed at least in part with a substantially free-form curve geometry.

Each side seam 630 intersects the waist opening edge 631 and one of the leg opening edges 633. At the intersection with the waist opening edge 631, a tangent to the centerline of the side seam 630 defines a first seam angle θ_{S1} relative to the vertical. The straight seam portion 636 defines a second seam angle with respect to the vertical at the intersection of the side seam 630 with the leg opening edge 633. In the embodiment illustrated in FIG. 13, the straight seam portion 636 is substantially vertical, thus making the second seam angle equal to 0 degrees. It will be understood, however, that in other embodiments, the straight seam portion 636 could be non-vertical, thus producing a non-zero second seam angle. The first seam angle θ_{S1} and the second seam angle may be designed specifically to accommodate different body types and sizes. For certain body types, the first and second seam angles may be equal. For other body types, the first and second seam angles may be significantly different. The first and second seam angles may each practicably be in a range of about 0 degrees to about 80 degrees. To accommodate the most typical body shapes, the first and second seam angles are each preferably in a range from about 10 degrees to about 50 degrees and most preferably in a range from about 25 degrees to about 35 degrees.

It will be understood that the final geometry of the waist opening side panel edges 670, 678 and the outer lateral side panel edges 672, 680 may be formed either before or after the side seam 630 is formed to join the first and second side portions 666, 674. In either case, the lateral side panel edges 672, 680 may be formed so as to substantially match the geometry of the side seam 630.

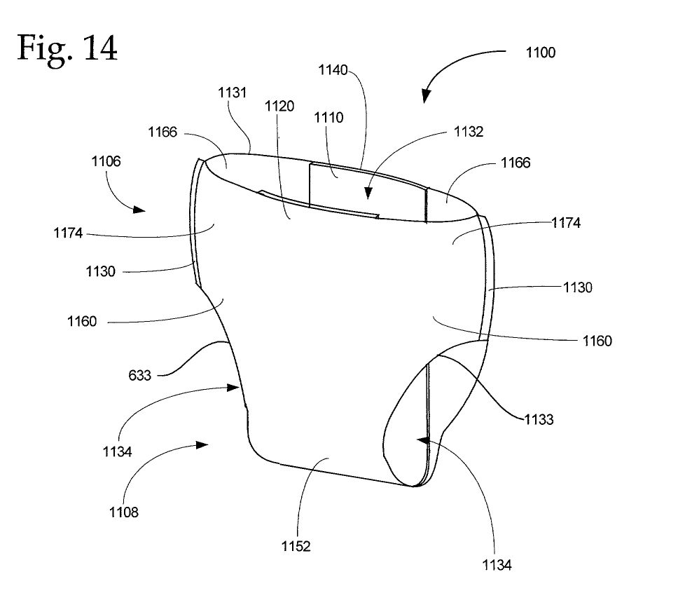

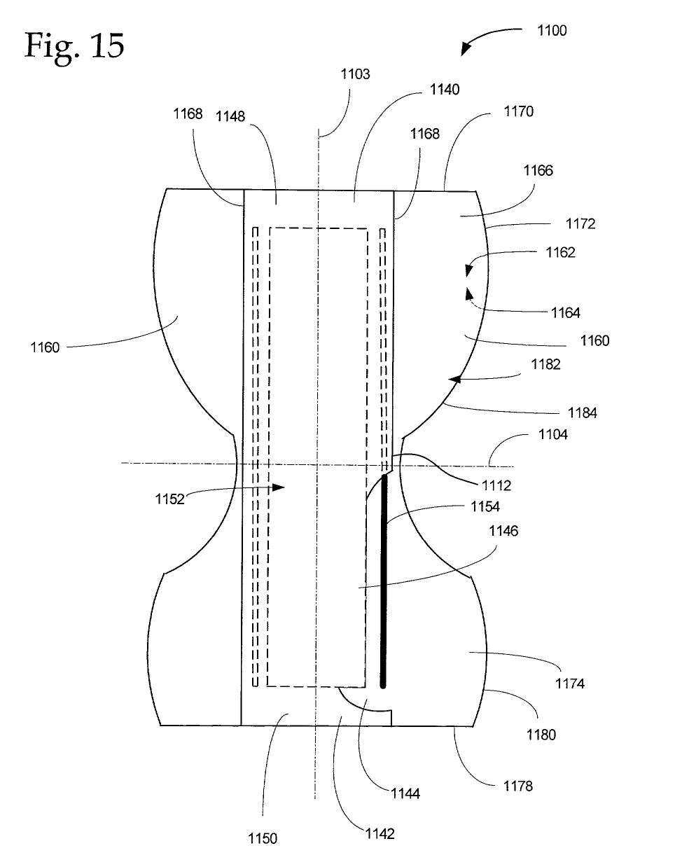

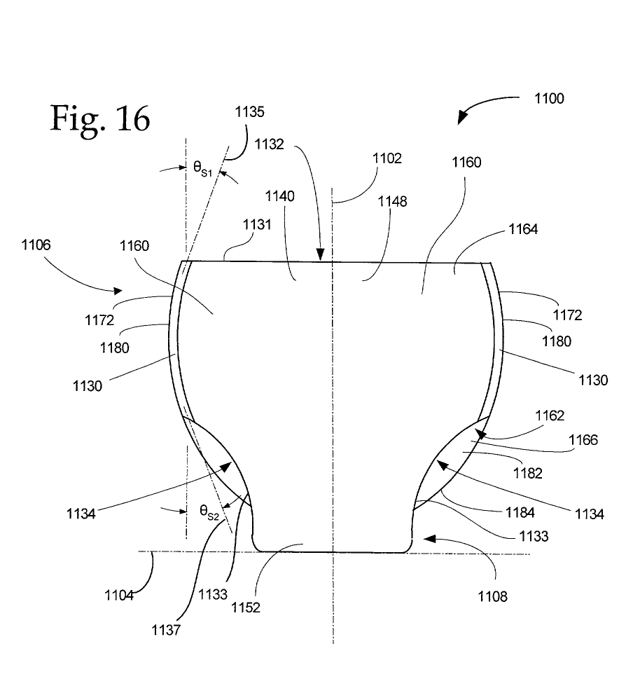

FIGS. 14-16 depict another preferred embodiment of an absorbent garment (preferably a disposable absorbent garment) of the present invention. The absorbent garment is depicted as and will be discussed as a child's training pant; however, this depiction is not intended to limit the invention, and those skilled in the art will appreciate that the invention covers other types of absorbent garments including incontinent pants for adults. The absorbent garment is depicted in FIGS. 14 and 16 in its fully assembled form as it would appear when worn, with FIG. 14 being a perspective view and FIG. 16 a front view. FIG. 15 illustrates the garment in an intermediate stage of assembly. At this stage, the garment is fully assembled except for the joinder at the side seams 1130, which will be discussed in more detail hereafter. In FIG. 15, the garment is shown in a generally flattened condition as it appears during an intermediate stage of construction.

The garment 1100 is shown with and will be described relative to first and second orthogonal axes 1102, 1104. The first axis 1102 may be referred to as the vertical axis 1102 and the second axis 1104 may be referred to as the lateral axis 1104. It will be understood by those having ordinary skill in the art that the vertical axis 1102 corresponds generally to a head-to-toe axis of a standing wearer and that the lateral axis 1104 corresponds generally to a side-to-side axis of a standing wearer.

FIG. 15 is a plan view of the body-facing side of the flattened garment 1100. In the flattened condition, the garment may have a generally hourglass shaped structure, but it may also have any other shape suitable for the given application. The flattened absorbent garment 1100 has a longitudinal axis 1103 extending from the portion of the garment that will conform to the front of the user to the portion of the garment that will conform to the rear of the user. The longitudinal axis 1103 of the flattened garment 1100 is orthogonal to the lateral axis 1104, which, as previously noted, extends from side to side relative to a wearer of the finished garment 1100.

In use, an aspect of the invention comprises a pant-like garment having a waist-encircling region 1106 and a crotch region 1108. The waist-encircling region 1106 may include a first waist portion 1110, disposable adjacent to, for example, the back waist area of a wearer's body, and a second waist portion 1120, disposable adjacent to, for example, the front waist area of a wearer's body. The first and second waist portions 1110, 1120 may therefore correspond to the back and front of the wearer's body, respectively. The first and second waist portions 1110, 1120 are joined to one another at side seams 1130 to form the waist encircling portion 1106 and to define a waist opening edge 1131 surrounding a waist opening 1132 and leg opening edges 1133 surrounding two leg openings 1134.

As in other embodiments of the invention, at least a portion of each side seam 1130 is curved so as to conform to a lower waist, hip or thigh contour of the wearer.

The absorbent garment 1100 comprises a generally planar main body 1140 folded or doubled to form a generally U-shaped structure. The main body 1140 preferably comprises a topsheet 1142 and a backsheet 1144. The topsheet 1142 and the backsheet 1144 may be substantially longitudinally coterminous. When the garment is being worn, the topsheet 1142 faces the wearer's body, and the backsheet 1144 faces away from the wearer. An absorbent core 1146 preferably is disposed between at least a portion of the topsheet 1142 and the backsheet 1144. The main body 1140 has a first main body waist portion 1148 configured to be disposable adjacent the back of the wearer's waist area and a second main body waist portion 1150 configured to be disposable adjacent the front of the wearer's waist area. The main body 1140 also has a central main body portion 1152 that connects the first and second main body waist portions 1148, 1150 and forms at least a portion of the crotch region 1108 when the main body 1140 is doubled.

The topsheet 1142 and backsheet 1144 may be constructed from a wide variety of materials known in the art. The invention is not intended to be limited to any specific materials for these components. The topsheet 1142 and backsheet can be shaped and sized according to the requirements of various types of absorbent garments, or to accommodate various user sizes. The topsheet 1142 and backsheet 1144 may, for example be configured to provide a main body 1140 that is hourglass shaped, rectangular, trapezoidal, “T” shaped, or other geometry.

Embodiments of the present invention may further comprise various additional features. For example, one or more pairs of elastic gathers 1154 may extend adjacent the lateral edges of the main body 1140. The main body 1140 may also comprise one or more waste containment systems, such as inboard standing leg gathers (not shown), which preferably extend from the second main body waist portion 1150 to the first main body waist portion 1148 along opposite sides of longitudinal center line 1102. One or both of the first and second main body waist portions 1148, 1150 may also be equipped with strips of elastic waist foam or other elastically extensible material (not shown), which help contract the garment around the wearer's waist, providing improved fit and leakage prevention.

The absorbent garment 1100 also includes side panels 1160 that extend outward from the two lateral edges 1168 of the main body 1140. In embodiments of the invention, the side panels 1160 may be integrally formed with a portion of the main body 1140, preferably the backsheet 1144. As shown in FIGS. 15, the backsheet 1144 may be a generally hourglass-shaped structure to which the other main body components are attached. The side panels 1160 may be formed using the portions of the backsheet 1144 that extend laterally outward beyond the lateral edges of the main body 1168.

Each side panel 1160 may be formed using only the backsheet 1144. Alternatively, an elasticized composite side panel 1160 may be formed by bonding additional layers to the backsheet 1144.

The elastic material of the side panels 1160 may include a single elastic or elasticized sheet or may be formed as an elasticized laminate.

The side panels 1160 each have a body facing surface 1162 and an opposing outward facing surface 1164. Each side panel 1160 preferably has a first side portion 1166 extending laterally outward from a main body lateral edge 1168 adjacent the first main body waist portion 1148 and a portion of the central main body portion 1152. The first side portion 1166 has a waist opening side panel edge 1170 and an outer lateral side panel edge 1172. Each side panel 1160 also has a second side portion 1174 extending laterally outward from the main body lateral edge 1168 adjacent the second main body waist portion 1150 and a portion of the central main body portion 1152. The second side portion 1174 also has a waist opening side panel edge 1178 and an outer lateral side panel edge 1180.

The first and second side portions 1166, 1174 are configured so that the body facing surface 1162 in the area adjacent the outer lateral side panel edge 1172 of the first side portion 1166 may be made to engage the body facing surface 1162 in the area adjacent the outer lateral side panel edge 1180 of the second side portion 1174 when the garment is folded to convert it from the configuration of FIG. 15 to the configuration of FIG. 16. With the body facing surface 1162 of the first and second side portions 1166, 1174 so-engaged, the first and second side portions 1166, 1174 may be joined by the formation of the side seam 1130 thereon. The first and second side seam portions 1166, 1174 are joined and the side seam 1130 is formed with the first and second side seam portions 1166,1174 in a fully stretched condition. As in other embodiments of the invention, the side seam 1130 may be formed using any method known in the art or a combination of such methods.

The first and second side portions 1166, 1174 may be configured so that at least a portion of the outer lateral side panel edge 1172 of the first side portion 1166 is a mirror image of at least a portion of the outer lateral side panel edge 1180 of the second side portion 1174. Further, the first and second side portions 1166, 1174 may be configured so that when the body facing surface 1162 of the first and second side portions 1166, 1174 is engaged by folding the garment during manufacture, the waist opening side panel edges 1170, 1178 can be substantially aligned and at least a portion of the outer lateral side panel edges 1172, 1180 can be substantially aligned.

When the first and second side portions 1166, 1174 of both side panels 1160 are joined, the first and second main body waist portions 1148,1150 combine with the side panels 1160 to form the waist encircling portion 1106, the waist opening edge 1131 and the waist opening 1132. Also, the central main body portion 1152 combines with the side panels 1160 to form the crotch region 1108, the leg opening edges 1133 and the two leg openings 1134. The curved side seam 1130 causes the completed side panels 1160 to adopt a pre-formed curvature that is not provided by previous garments having straight seams or seams formed only from straight segments.

As shown in FIGS. 14-16, the side seam 1130 may be curved along its entire length. While the illustrated curve of the side seam is a circular arc segment, it will be understood that the present invention is not limited to a particular curve geometry. The side seam 1130 of embodiments of the present invention may use any curvature that serves to enhance comfort and fit by better conformance with body curves of the wearer. For example, the geometry of the side seam 1130 may include other types of regular curve segments such as an ellipse or can be formed from a plurality of similar or disparate regular curve segments. The side seam 1130 can also be formed at least in part with a substantially free-form curve geometry.

Each side seam 1130 intersects the waist opening edge 1131 and one of the leg opening edges 1133. At the intersection with the waist opening edge 1131, a tangent to the centerline of the side seam 1130 defines a first seam angle θ_{S1} relative to the vertical. At the intersection with the leg opening edge 1133, a tangent to the centerline of the side seam 1130 defines a second seam angle θ_{S2} relative to the vertical. The first and second seam angles θ_{S1}, θ_{S2} may be designed specifically to accommodate different body types and sizes. For certain body types, the first and second seam angles θ_{S1}, θ_{S2} may be equal. For other body types, the first and second seam angles θ_{S1}, θ_{S2} may be significantly different. The first and second seam angles θ_{S1}, θ_{S2} may each practicably be in a range of about 0 degrees to about 80 degrees. To accommodate the most typical body shapes, the first and second seam angles θ_{S1}, θ_{S2} are each preferably in a range from about 10 degrees to about 50 degrees and most preferably in a range from about 25 degrees to about 35 degrees.

It will be understood that the final geometry of the waist opening side panel edges 1170, 1178 and the outer lateral side panel edges 1172, 1180 may be formed either before or after the side seam 1130 is formed to join the first and second side portions 1166, 1174. In either case, the lateral side panel edges 1172, 1180 may be formed so as to substantially match the geometry of the side seam 1130.

The first side portion 1166 may include a lower hip region 1182 that provides additional coverage of the back of the wearer in the area adjacent the wearer's hip region. As best seen in FIGS. 15 and 16, this area extends laterally outward and downward beyond the corresponding area of the second side portion 1174. As shown, the lateral edge 1184 of the lower hip region 1182 is curved. For comfort and aesthetic reasons, the curvature of the lateral edge 1184 may be a continuation of the curvature of the outer lateral side panel edge 1172. It will be understood, however, that the lateral edge 1184 need not have a continuous curve and, in fact, need not be curved at all.

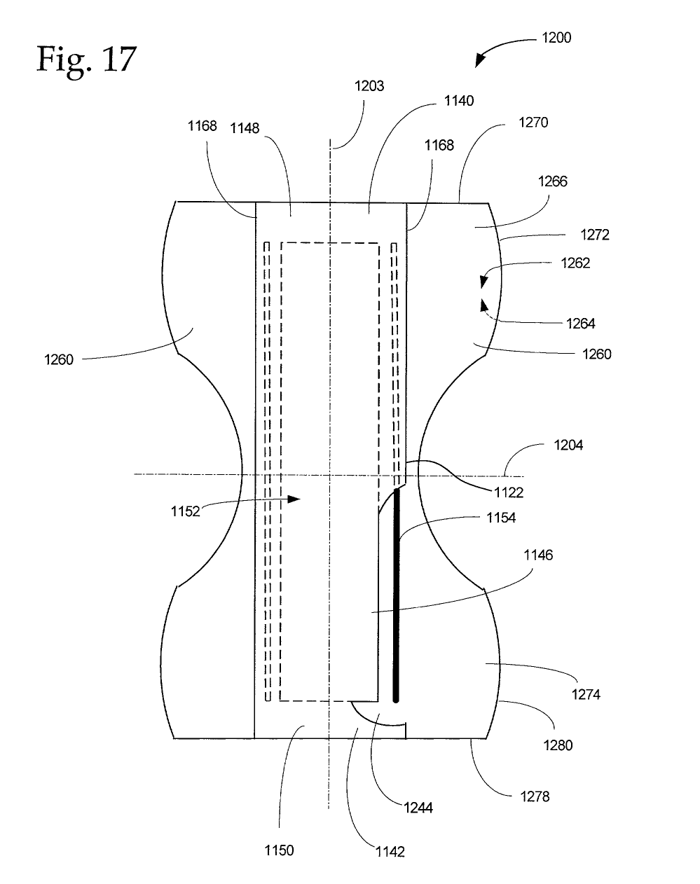

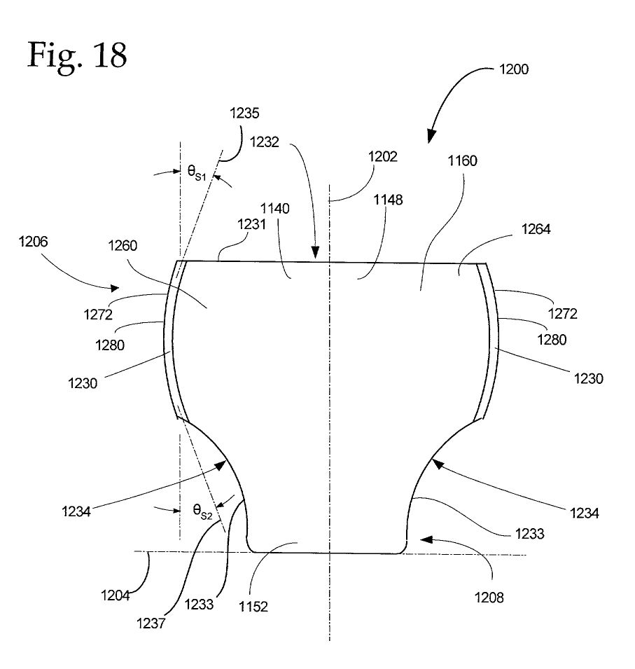

In an alternative embodiment of the invention illustrated in FIGS. 17 and 18, an absorbent garment 1200 is provided that is similar to the absorbent garment 1100 of FIGS. 14-16. The first side portions 1266 of the absorbent garment 1200, however, do not include an extended lower hip portion. As shown in FIG. 17, this may result in a substantially symmetric hourglass-shaped backsheet 1244.

A front view of the absorbent garment 1200 in its fully assembled form as it would appear when worn is shown in FIG. 18. FIG. 17 illustrates the garment 1200 in an intermediate stage of assembly. At this stage, the garment 1200 is fully assembled except for the joinder at the side seams 1230. In FIG. 17, the garment 1200 is shown in a generally flattened condition as it appears during the intermediate stage of assembly.

The garment 1200 is shown with and will be described relative to first and second orthogonal axes 1202, 1204. The first axis 1202 may be referred to as the vertical axis 1202 and the second axis 1204 may be referred to as the lateral axis 1204. It will be understood by those having ordinary skill in the art that the vertical axis 1202 corresponds generally to a head-to-toe axis of a standing wearer and that the lateral axis 1204 corresponds generally to a side-to-side axis of a standing wearer.

FIG. 17 is a plan view of the body-facing side of the flattened garment 1200. In the flattened condition, the garment 1200 may have a generally symmetric hourglass shaped structure, but it may also have any other shape suitable for the given application. The flattened absorbent garment 1200 has a longitudinal axis 1203 extending from the portion of the garment 1200 that will conform to the front of the user to the portion of the garment that will conform to the rear of the user. The longitudinal axis 1203 of the flattened garment 1200 is orthogonal to the lateral axis 1204, which, as previously noted, extends from side to side relative to a wearer of the finished garment 1200.

The absorbent garment 1200 may have a main body 1140 and associated components that, with the exception of the backsheet 1244, are substantially similar to those of the absorbent garment 1100 of FIGS. 14-16. The absorbent garment 1200 includes side panels 1260 extending laterally outward from the main body lateral edges 1168. The side panels 1260 may be formed at least in part from the backsheet 1244. The side panels 1260 may be formed using only the backsheet 1244 or an elasticized composite side panel 1260 may be formed by bonding additional layers to the backsheet 1244.

Each side panel 1260 has a first side portion 1266 extending laterally outward from a main body lateral edge 1168 adjacent the first main body waist portion 1148 and a portion of the central main body portion 1152. The first side portion 1266 has a waist opening side panel edge 1270 and an outer lateral side panel edge 1272. Each side panel 1260 also has a second side portion 1274 extending laterally outward from the main body lateral edge 1168 adjacent the second main body waist portion 1150 and a portion of the central main body portion 1152. The second side portion 1274 also has a waist opening side panel edge 1278 and an outer lateral side panel edge 1280.

The first and second side portions 1266, 1274 are configured so that the body facing surface 1262 in the area adjacent the outer lateral side panel edge 1272 of the first side portion 1266 may be made to engage the body facing surface 1262 in the area adjacent the outer lateral side panel edge 1280 of the second side portion 1274 when the garment is folded to convert it from the configuration of FIG. 17 to the configuration of FIG. 18. With the body facing surface 1262 of the first and second side portions 1266, 1274 so-engaged, the first and second side portions 1266, 1274 may be joined by the formation of the side seam 1230 thereon. The first and second side seam portions 1266, 1274 are joined and the side seam 1230 is formed with the first and second side seam portions 1266, 1274 in a fully stretched condition.

The first and second side portions 1266, 1274 may be configured so that at least a portion of the outer lateral side panel edge 1272 of the first side portion 1266 is a mirror image of at least a portion of the outer lateral side panel edge 1280 of the second side portion 1274. Further, the first and second side portions 1266, 1274 may be configured so that when the body facing surface 1262 of the first and second side portions 1266, 1274 is engaged by folding the garment during manufacture, the waist opening side panel edges 1270, 1278 can be substantially aligned and at least a portion of the outer lateral side panel edges 1272, 1280 can be substantially aligned.

When the first and second side portions 1266, 1274 of both side panels 1260 are joined, the first and second main body waist portions 1148, 1150 combine with the side panels 1260 to form a waist encircling portion 1206, a waist opening edge 1231 and a waist opening 1232. Also, the central main body portion 1152 combines with the side panels 1260 to form a crotch region 1208, leg opening edges 1233 and two leg openings 1234. The curved side seam 1230 causes the completed side panels 1260 to adopt a pre-formed curvature that is not provided by previous garments having straight seams or seams formed only from straight segments.

The side seam 1230 may be curved along its entire length. While the illustrated curve of the side seam is a circular arc segment, it will be understood that the present invention is not limited to a particular curve geometry. The side seam 1230 of embodiments of the present invention may use any curvature that serves to enhance comfort and fit by better conformance with body curves of the wearer. For example, the geometry of the side seam 1230 may include other types of regular curve segments such as an ellipse or can be formed from a plurality of similar or disparate regular curve segments. The side seam 1230 can also be formed at least in part with a substantially free-form curve geometry.

Each side seam 1230 intersects the waist opening edge 1231 and one of the leg opening edges 1233. At the intersection with the waist opening edge 1231, a tangent to the centerline of the side seam 1230 defines a first seam angle θ_{S1} relative to the vertical. At the intersection with the leg opening edge 1233, a tangent to the centerline of the side seam 1230 defines a second seam angle θ_{S2} relative to the vertical. The first and second seam angles θ_{S1}, θ_{S2} may be designed specifically to accommodate different body types and sizes. For certain body types, the first and second seam angles θ_{S1}, θ_{S2} may be equal. For other body types, the first and second seam angles θ_{S1}, θ_{S2} may be significantly different. The first and second seam angles θ_{S1}, θ_{S2} may each practicably be in a range of about 0 degrees to about 80 degrees. To accommodate the most typical body shapes, the first and second seam angles θ_{S1}, θ_{S2} are each preferably in a range from about 10 degrees to about 50 degrees and most preferably in a range from about 25 degrees to about 35 degrees.

It will be understood that the final geometry of the waist opening side panel edges 1270, 1278 and the outer lateral side panel edges 1272, 1280 may be formed either before or after the side seam 1230 is formed to join the first and second side portions 1266, 1274. In either case, the lateral side panel edges 1272, 1280 may be formed so as to substantially match the geometry of the side seam 1230.

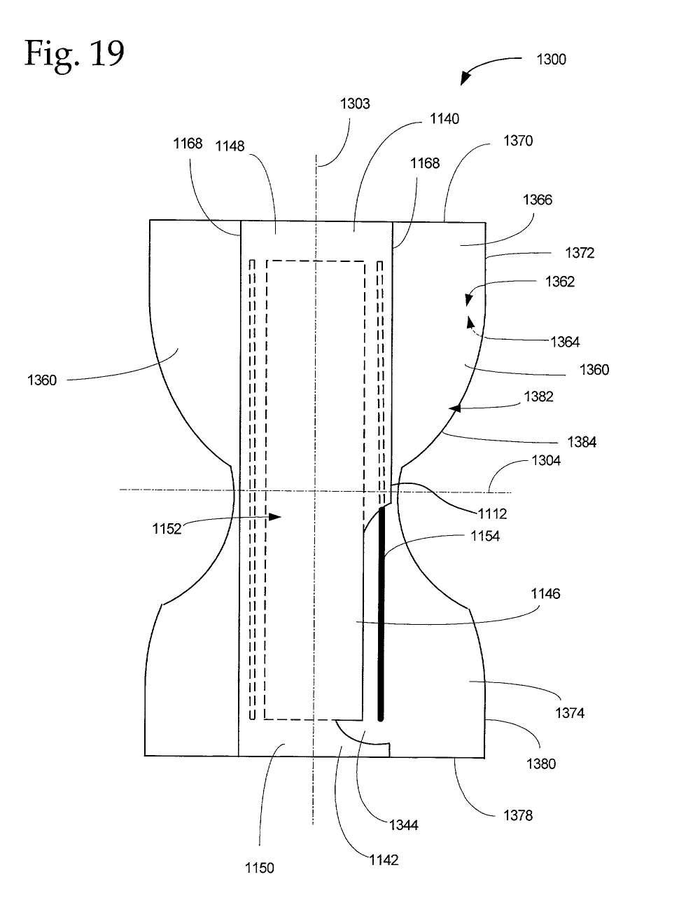

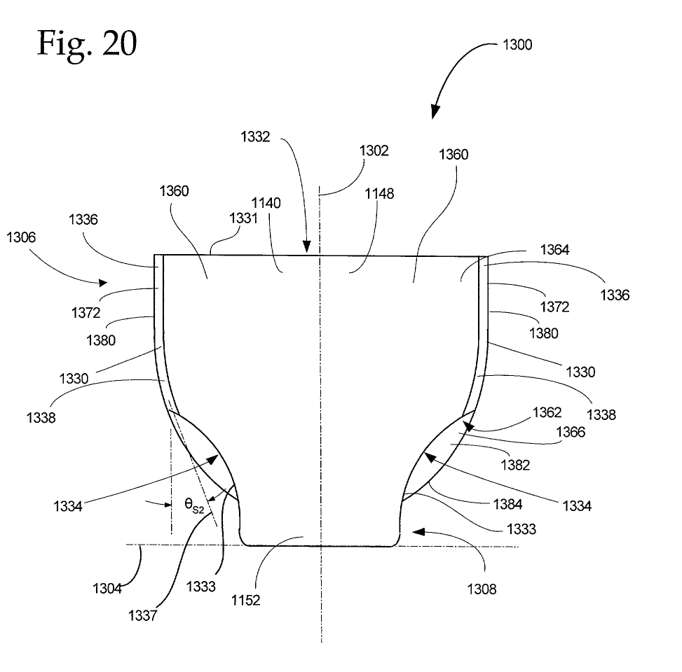

As previously discussed, for certain wearers, it may not be necessary to provide a garment with side seams that are curved over their entire length. Accordingly, FIGS. 19 and 20 depict an exemplary embodiment of an absorbent garment 1300 wherein only a portion of the side seam is curved. In the absorbent garment 1300, the first and second side portions 1366, 1374 are joined by forming a side seam 1330 that is substantially straight in the area adjacent the waist opening 1132 and curved in the area adjacent the leg openings 1334. This configuration may provide additional comfort and security to a wearer having, for example, a disproportionately small waist as compared to the wearer's hips and thighs.

A front view of the absorbent garment 1300 in its fully assembled form as it would appear when worn is shown in FIG. 20. FIG. 19 illustrates the garment 1300 in an intermediate stage of assembly. At this stage, the garment 1300 is fully assembled except for the joinder at the side seams 1330. In FIG. 19, the garment 1300 is shown in a generally flattened condition as it appears during the intermediate stage of assembly.

The garment 1300 is shown with and will be described relative to first and second orthogonal axes 1302, 1304. The first axis 1302 may be referred to as the vertical axis 1302 and the second axis 1304 may be referred to as the lateral axis 1304. It will be understood by those having ordinary skill in the art that the vertical axis 1302 corresponds generally to a head-to-toe axis of a standing wearer and that the lateral axis 1304 corresponds generally to a side-to-side axis of a standing wearer.

FIG. 19 is a plan view of the body-facing side of the flattened garment 1300. In the flattened condition, the garment 1300 may have a generally hourglass shaped structure, but it may also have any other shape suitable for the given application. The flattened absorbent garment 1300 has a longitudinal axis 1303 extending from the portion of the garment 1300 that will conform to the front of the user to the portion of the garment that will conform to the rear of the user. The longitudinal axis 1303 of the flattened garment 1300 is orthogonal to the lateral axis 1304, which, as previously noted, extends from side to side relative to a wearer of the finished garment 1300.

The absorbent garment 1300 may have a main body 1140 and associated components that, with the exception of the backsheet 1344, are substantially similar to those of the absorbent garment 1100 of FIGS. 14-16. The absorbent garment 1300 includes side panels 1360 extending laterally outward from the main body lateral edges 1168. The side panels 1360 may be formed at least in part from the backsheet 1344. The side panels 1360 may be formed using only the backsheet 1344 or an elasticized composite side panel 1360 may be formed by bonding additional layers to the backsheet 1344.

Each side panel 1360 has a first side portion 1366 extending laterally outward from a main body lateral edge 1168 adjacent the first main body waist portion 1148 and a portion of the central main body portion 1152. The first side portion 1366 has a waist opening side panel edge 1370 and an outer lateral side panel edge 1372. Each side panel 1360 also has a second side portion 1374 extending laterally outward from the main body lateral edge 1168 adjacent the second main body waist portion 1150 and a portion of the central main body portion 1152. The second side portion 1374 also has a waist opening side panel edge 1378 and an outer lateral side panel edge 1380.

The first and second side portions 1366, 1374 are configured so that the body facing surface 1362 in the area adjacent the outer lateral side panel edge 1372 of the first side portion 1366 may be made to engage the body facing surface 1362 in the area adjacent the outer lateral side panel edge 1380 of the second side portion 1374 when the garment is folded to convert it from the configuration of FIG. 19 to the configuration of FIG. 20. With the body facing surface 1362 of the first and second side portions 1366, 1374 so-engaged, the first and second side portions 1366, 1374 may be joined by the formation of the side seam 1330 thereon. The first and second side seam portions 1366, 1374 are joined and the side seam 1330 is formed with the first and second side seam portions 1366, 1374 in a fully stretched condition.

The first and second side portions 1366, 1374 may be configured so that at least a portion of the outer lateral side panel edge 1372 of the first side portion 1366 is a mirror image of at least a portion of the outer lateral side panel edge 1380 of the second side portion 1374. Further, the first and second side portions 1366, 1374 may be configured so that when the body facing surface 1362 of the first and second side portions 1366, 1374 is engaged by folding the garment during manufacture, the waist opening side panel edges 1370, 1378 can be substantially aligned and at least a portion of the outer lateral side panel edges 1372, 1380 can be substantially aligned.

When the first and second side portions 1366, 1374 of both side panels 1360 are joined, the first and second main body waist portions 1148, 1150 combine with the side panels 1360 to form a waist encircling portion 1306, a waist opening edge 1331 and a waist opening 1332. Also, the central main body portion 1152 combines with the side panels 1360 to form a crotch region 1308, leg opening edges 1333 and two leg openings 1334. The partially curved side seam 1330 causes the completed side panels 1360 to adopt a pre-formed curvature that is not provided by previous garments having straight seams or seams formed only from straight segments.

In this embodiment, each side seam 1330 has a straight portion 1336 adjacent the waist opening 1332 and a curved portion 1338 adjacent the leg opening 1334 on the same lateral side of the garment 1300 as the side seam 1330. The curved portion 1338 of side seam 1330 may have any curvature that serves to enhance comfort and fit by better conformance with body curves of the wearer. For example, the geometry of the curved portion 1338 may include regular curve segments such as circular or elliptical arcs or can be formed from a plurality of similar or disparate regular curve segments. The curved portion 1338 may also be formed at least in part with a substantially free-form curve geometry.

Each side seam 1330 intersects the waist opening edge 1331 and one of the leg opening edges 1333. The straight seam portion 1336 defines a first seam angle with respect to the vertical at the intersection of the side seam 1330 with the waist opening edge 1331. In the embodiment illustrated in FIG. 20, the straight seam portion 1336 is substantially vertical, thus making the first seam angle equal to 0 degrees. It will be understood, however, that in other embodiments, the straight seam portion 1336 could be non-vertical, thus producing a non-zero first seam angle. At the intersection with the leg opening edge 1333, a tangent to the centerline of the curved seam portion 1338 defines a second seam angle θ_{S2} relative to the vertical. The first seam angle and the second seam angle θ_{S2} may be designed specifically to accommodate different body types and sizes. For certain body types, the first and second seam angles may be equal. For other body types, the first and second seam angles may be significantly different. The first and second seam angles may each practicably be in a range of about 0 degrees to about 80 degrees. To accommodate the most typical body shapes, the first and second seam angles are each preferably in a range from about 10 degrees to about 50 degrees and most preferably in a range from about 25 degrees to about 35 degrees.

It will be understood that the final geometry of the waist opening side panel edges 1370, 1378 and the outer lateral side panel edges 1372, 1380 may be formed either before or after the side seam 1330 is formed to join the first and second side portions 1366, 1374. In either case, the lateral side panel edges 1372, 1380 may be formed so as to substantially match the geometry of the side seam 1330.

The first side portions 1366 of the absorbent garment 1300 each include a lower hip region 1382 that is similar to that described above for the absorbent garment 1100 of FIGS. 14-16. As shown in FIGS. 19 and 20, the lateral edge 1384 of the lower hip region 1382 is curved. For comfort and aesthetic reasons, the curvature of the lateral edge 1384 may be a continuation of the curvature of the outer lateral side panel edge 1372. It will be understood, however, that the lateral edge 1384 need not have a continuous curve and, in fact, need not be curved at all.

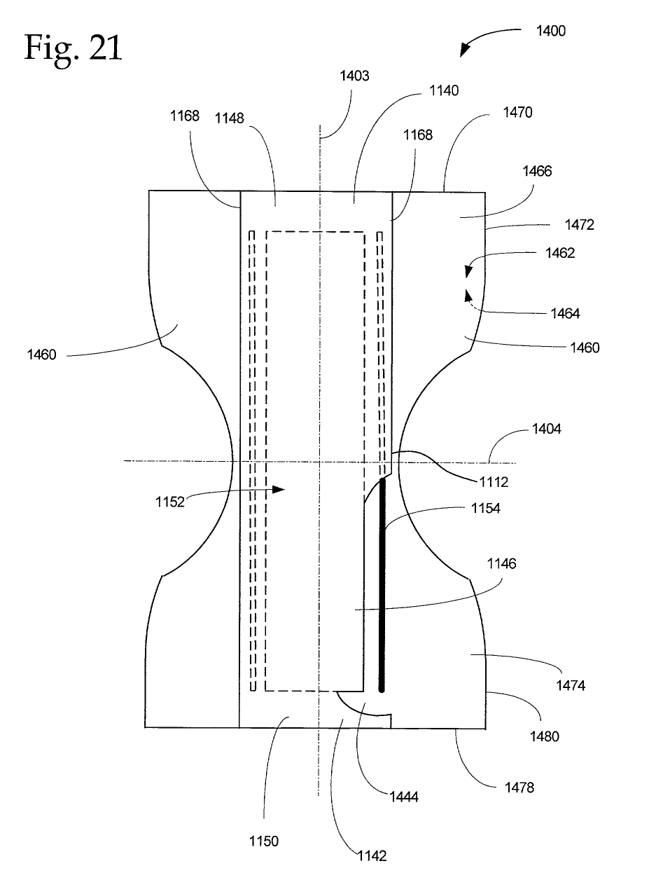

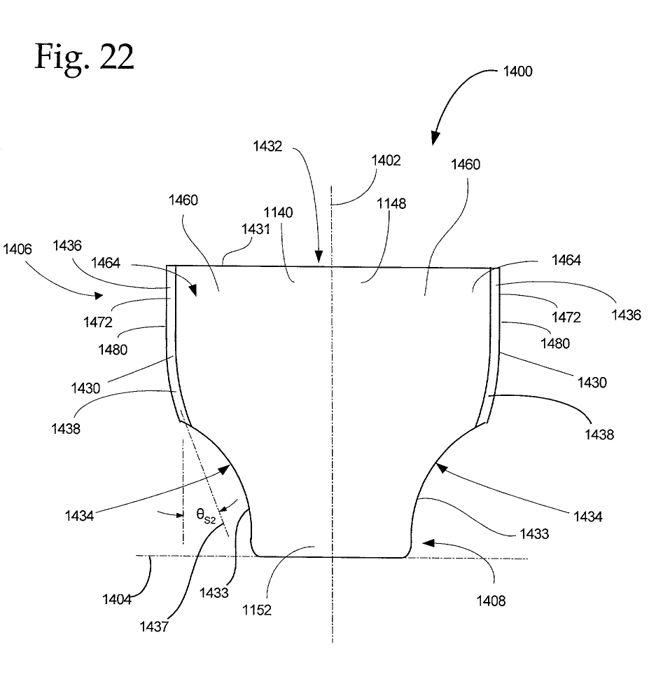

FIGS. 21 and 22 depict a similar absorbent garment 1400 having first side portions 1466 that do not have the lower hip region. A front view of the absorbent garment 1400 in its fully assembled form as it would appear when worn is shown in FIG. 22. FIG. 21 illustrates the garment 1400 in an intermediate stage of assembly. At this stage, the garment 1400 is fully assembled except for the joinder at the side seams 1430. In FIG. 21, the garment 1400 is shown in a generally flattened condition as it appears during the intermediate stage of assembly.

The garment 1400 is shown with and will be described relative to first and second orthogonal axes 1402, 1404. The first axis 1402 may be referred to as the vertical axis 1402 and the second axis 1404 may be referred to as the lateral axis 1404. It will be understood by those having ordinary skill in the art that the vertical axis 1402 corresponds generally to a head-to-toe axis of a standing wearer and that the lateral axis 1404 corresponds generally to a side-to-side axis of a standing wearer.

FIG. 21 is a plan view of the body-facing side of the flattened garment 1400. In the flattened condition, the garment 1400 may have a generally symmetric hourglass shaped structure, but it may also have any other shape suitable for the given application. The flattened absorbent garment 1400 has a longitudinal axis 1403 extending from the portion of the garment 1400 that will conform to the front of the user to the portion of the garment that will conform to the rear of the user. The longitudinal axis 1403 of the flattened garment 1400 is orthogonal to the lateral axis 1404, which, as previously noted, extends from side to side relative to a wearer of the finished garment 1400.

The absorbent garment 1400 may have a main body 1140 and associated components that, with the exception of the backsheet 1444, are substantially similar to those of the absorbent garment 1100 of FIGS. 14-16. The absorbent garment 1400 includes side panels 1460 extending laterally outward from the main body lateral edges 1168. The side panels 1460 may be formed at least in part from the backsheet 1444. The side panels 1460 may be formed using only the backsheet 1444 or an elasticized composite side panel 1460 may be formed by bonding additional layers to the backsheet 1444.

Each side panel 1460 has a first side portion 1466 extending laterally outward from a main body lateral edge 1168 adjacent the first main body waist portion 1148 and a portion of the central main body portion 1152. The first side portion 1466 has a waist opening side panel edge 1470 and an outer lateral side panel edge 1472. Each side panel 1460 also has a second side portion 1474 extending laterally outward from the main body lateral edge 1168 adjacent the second main body waist portion 1150 and a portion of the central main body portion 1152. The second side portion 1474 also has a waist opening side panel edge 1478 and an outer lateral side panel edge 1480.

The first and second side portions 1466, 1474 are configured so that the body facing surface 1462 in the area adjacent the outer lateral side panel edge 1472 of the first side portion 1466 may be made to engage the body facing surface 1462 in the area adjacent the outer lateral side panel edge 1480 of the second side portion 1474 when the garment is folded to convert it from the configuration of FIG. 21 to the configuration of FIG. 22. With the body facing surface 1462 of the first and second side portions 1466, 1474 so-engaged, the first and second side portions 1466, 1474 may be joined by the formation of the side seam 1430 thereon. The first and second side seam portions 1466, 1474 are joined and the side seam 1430 is formed with the first and second side seam portions 1466, 1474 in a fully stretched condition.

The first and second side portions 1466, 1474 may be configured so that at least a portion of the outer lateral side panel edge 1472 of the first side portion 1466 is a mirror image of at least a portion of the outer lateral side panel edge 1480 of the second side portion 1474. Further, the first and second side portions 1466, 1474 may be configured so that when the body facing surface 1462 of the first and second side portions 1466, 1474 is engaged by folding the garment during manufacture, the waist opening side panel edges 1470, 1478 can be substantially aligned and at least a portion of the outer lateral side panel edges 1472, 1480 can be substantially aligned.

When the first and second side portions 1466, 1474 of both side panels 1460 are joined, the first and second main body waist portions 1148, 1150 combine with the side panels 1460 to form a waist encircling portion 1406, a waist opening edge 1431 and a waist opening 1432. Also, the central main body portion 1152 combines with the side panels 1460 to form a crotch region 1408, leg opening edges 1433 and two leg openings 1434. The partially curved side seam 1430 causes the completed side panels 1460 to adopt a pre-formed curvature that is not provided by previous garments having straight seams or seams formed only from straight segments.

In this embodiment, each side seam 1430 has a straight portion 1436 adjacent the waist opening 1432 and a curved portion 1438 adjacent the leg opening 1434 on the same lateral side of the garment 1400 as the side seam 1430. The curved portion 1438 of side seam 1430 may have any curvature that serves to enhance comfort and fit by better conformance with body curves of the wearer. For example, the geometry of the curved portion 1438 may include regular curve segments such as circular or elliptical arcs or can be formed from a plurality of similar or disparate regular curve segments. The curved portion 1438 may also be formed at least in part with a substantially free-form curve geometry.

Each side seam 1430 intersects the waist opening edge 1431 and one of the leg opening edges 1433. The straight seam portion 1436 defines a first seam angle with respect to the vertical at the intersection of the side seam 1430 with the waist opening edge 1431. In the embodiment illustrated in FIG. 22, the straight seam portion 1436 is substantially vertical, thus making the first seam angle equal to 0 degrees. It will be understood, however, that in other embodiments, the straight seam portion 1436 could be non-vertical, thus producing a non-zero first seam angle. At the intersection with the leg opening edge 1433, a tangent to the centerline of the curved seam portion 1438 defines a second seam angle θ_{S2} relative to the vertical. The first seam angle and the second seam angle θ_{S2} may be designed specifically to accommodate different body types and sizes. For certain body types, the first and second seam angles may be equal. For other body types, the first and second seam angles may be significantly different. The first and second seam angles may each practicably be in a range of about 0 degrees to about 80 degrees. To accommodate the most typical body shapes, the first and second seam angles are each preferably in a range from about 10 degrees to about 50 degrees and most preferably in a range from about 25 degrees to about 35 degrees.

It will be understood that the final geometry of the waist opening side panel edges 1470, 1478 and the outer lateral side panel edges 1472, 1480 may be formed either before or after the side seam 1430 is formed to join the first and second side portions 1466, 1474. In either case, the lateral side panel edges 1472, 1480 may be formed so as to substantially match the geometry of the side seam 1430.

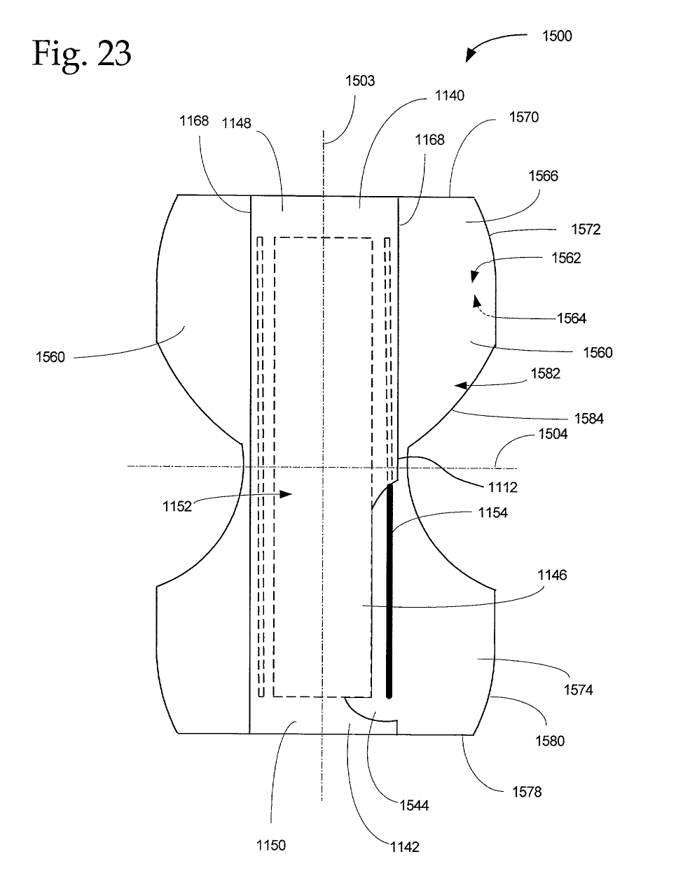

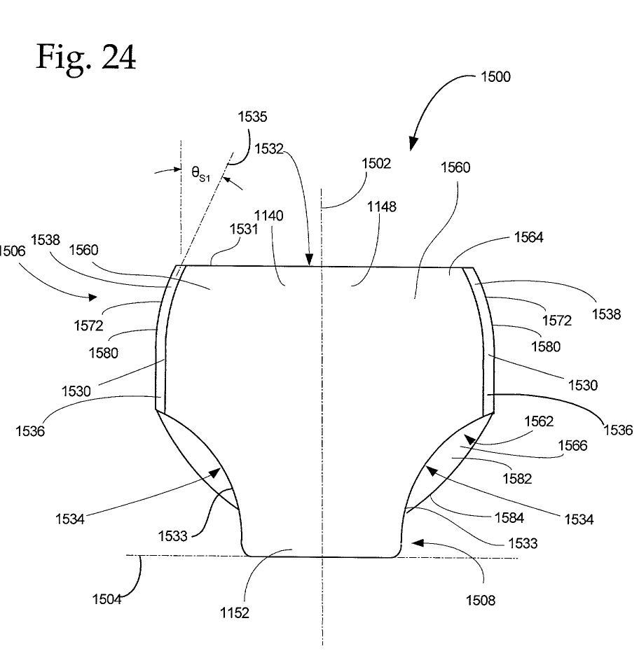

In another exemplary embodiment depicted in FIGS. 23 and 24, an absorbent garment 1500 has first and second side portions 1566, 1574 that are joined by forming a side seam 1530 that is substantially straight in the areas adjacent the leg openings 1534 and curved in the area adjacent the waist opening 1532. This configuration may provide additional comfort and leakage security in wearers having disproportionately small thighs as compared to the wearer's waist. The first side portions 1566 of the garment 1500 each include a lower hip region 1582 that is similar to that described above for the absorbent garment 1100 of FIGS. 14-16.

A front view of the absorbent garment 1500 in its fully assembled form as it would appear when worn is shown in FIG. 24. FIG. 23 illustrates the garment 1500 in an intermediate stage of assembly. At this stage, the garment 1500 is fully assembled except for the joinder at the side seams 1530. In FIG. 23, the garment 1500 is shown in a generally flattened condition as it appears during the intermediate stage of assembly.

The garment 1500 is shown with and will be described relative to first and second orthogonal axes 1502, 1504. The first axis 1502 may be referred to as the vertical axis 1502 and the second axis 1504 may be referred to as the lateral axis 1504. It will be understood by those having ordinary skill in the art that the vertical axis 1502 corresponds generally to a head-to-toe axis of a standing wearer and that the lateral axis 1504 corresponds generally to a side-to-side axis of a standing wearer.

FIG. 23 is a plan view of the body-facing side of the flattened garment 1500. In the flattened condition, the garment 1500 may have a generally hourglass shaped structure, but it may also have any other shape suitable for the given application. The flattened absorbent garment 1500 has a longitudinal axis 1503 extending from the portion of the garment 1500 that will conform to the front of the user to the portion of the garment that will conform to the rear of the user. The longitudinal axis 1503 of the flattened garment 1500 is orthogonal to the lateral axis 1504, which, as previously noted, extends from side to side relative to a wearer of the finished garment 1500.

The absorbent garment 1500 may have a main body 1140 and associated components that, with the exception of the backsheet 1244, are substantially similar to those of the absorbent garment 1100 of FIGS. 14-16. The absorbent garment 1500 includes side panels 1560 extending laterally outward from the main body lateral edges 1168. The side panels 1560 may be formed at least in part from the backsheet 1544. The side panels 1560 may be formed using only the backsheet 1544 or an elasticized composite side panel 1560 may be formed by bonding additional layers to the backsheet 1544.

Each side panel 1560 has a first side portion 1566 extending laterally outward from a main body lateral edge 1168 adjacent the first main body waist portion 1148 and a portion of the central main body portion 1152. The first side portion 1566 has a waist opening side panel edge 1570 and an outer lateral side panel edge 1572. Each side panel 1560 also has a second side portion 1574 extending laterally outward from the main body lateral edge 1168 adjacent the second main body waist portion 1150 and a portion of the central main body portion 1152. The second side portion 1574 also has a waist opening side panel edge 1578 and an outer lateral side panel edge 1580.

The first and second side portions 1566, 1574 are configured so that the body facing surface 1562 in the area adjacent the outer lateral side panel edge 1572 of the first side portion 1566 may be made to engage the body facing surface 1562 in the area adjacent the outer lateral side panel edge 1580 of the second side portion 1574 when the garment is folded to convert it from the configuration of FIG. 23 to the configuration of FIG. 24. With the body facing surface 1562 of the first and second side portions 1566, 1574 so-engaged, the first and second side portions 1566, 1574 may be joined by the formation of the side seam 1530 thereon. The first and second side seam portions 1566, 1574 are joined and the side seam 1530 is formed with the first and second side seam portions 1566, 1574 in a fully stretched condition.

The first and second side portions 1566, 1574 may be configured so that at least a portion of the outer lateral side panel edge 1572 of the first side portion 1566 is a mirror image of at least a portion of the outer lateral side panel edge 1580 of the second side portion 1574. Further, the first and second side portions 1566, 1574 may be configured so that when the body facing surface 1562 of the first and second side portions 1566, 1574 is engaged by folding the garment during manufacture, the waist opening side panel edges 1570, 1578 can be substantially aligned and at least a portion of the outer lateral side panel edges 1572, 1580 can be substantially aligned.

When the first and second side portions 1566, 1574 of both side panels 1560 are joined, the first and second main body waist portions 1148, 1150 combine with the side panels 1560 to form a waist encircling portion 1506, a waist opening edge 1531 and a waist opening 1532. Also, the central main body portion 1152 combines with the side panels 1560 to form a crotch region 1508, leg opening edges 1533 and two leg openings 1534. The partially curved side seam 1530 causes the completed side panels 1560 to adopt a pre-formed curvature that is not provided by previous garments having straight seams or seams formed only from straight segments.

In this embodiment, each side seam 1530 has a straight portion 1536 adjacent the leg opening 1534 on the same lateral side of the garment 1500 as the side seam 1530 and a curved portion 1538 adjacent the waist opening 1532. The curved portion 1538 of side seam 1530 may have any curvature that serves to enhance comfort and fit by better conformance with body curves of the wearer. For example, the geometry of the curved portion 1538 may include regular curve segments such as circular or elliptical arcs or can be formed from a plurality of similar or disparate regular curve segments. The curved portion 1538 may also be formed at least in part with a substantially free-form curve geometry.

Each side seam 1530 intersects the waist opening edge 1531 and one of the leg opening edges 1533. At the intersection with the waist opening edge 1531, a tangent to the centerline of the side seam 1530 defines a first seam angle θ_{S1} relative to the vertical. The straight seam portion 1536 defines a second seam angle with respect to the vertical at the intersection of the side seam 1530 with the leg opening edge 1533. In the embodiment illustrated in FIG. 11, the straight seam portion 1536 is substantially vertical, thus making the second seam angle equal to 0 degrees. It will be understood, however, that in other embodiments, the straight seam portion 1536 could be non-vertical, thus producing a non-zero second seam angle. The first seam angle θ_{S1} and the second seam angle may be designed specifically to accommodate different body types and sizes. For certain body types, the first and second seam angles may be equal. For other body types, the first and second seam angles may be significantly different. The first and second seam angles may each practicably be in a range of about 0 degrees to about 80 degrees. To accommodate the most typical body shapes, the first and second seam angles are each preferably in a range from about 10 degrees to about 50 degrees and most preferably in a range from about 25 degrees to about 35 degrees.

It will be understood that the final geometry of the waist opening side panel edges 1570, 1578 and the outer lateral side panel edges 1572, 1580 may be formed either before or after the side seam 1530 is formed to join the first and second side portions 1566, 1574. In either case, the lateral side panel edges 1572, 1580 may be formed so as to substantially match the geometry of the side seam 1530.

The first side portion 1566 includes a lower hip region 1582 that provides additional coverage of the back of the wearer in the area adjacent the wearer's hip region. This area extends laterally outward and downward beyond the corresponding area of the second side portion 1574. Although the lateral edge 1584 of the lower hip region 1182 is shown as curved, it will be understood that the lateral edge 1584 may be straight or a combination of curved and straight.

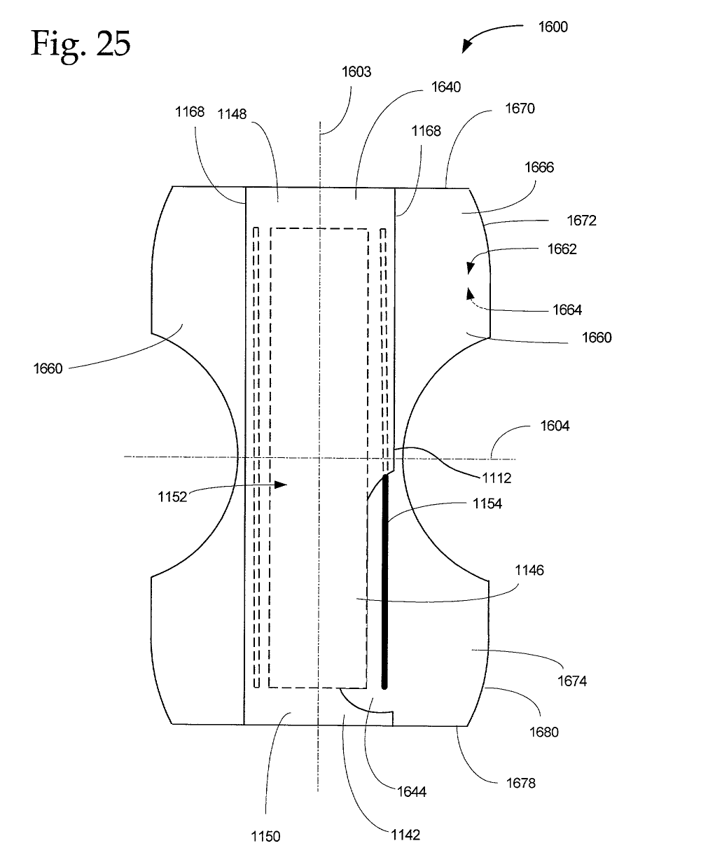

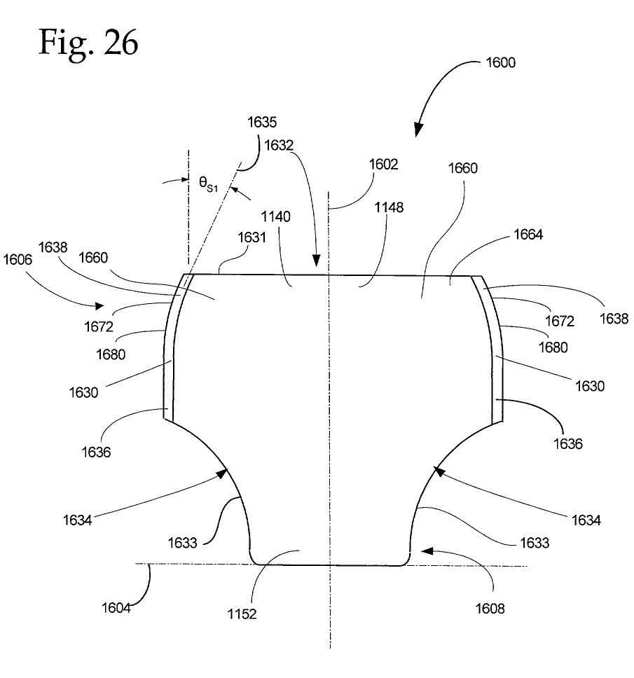

FIGS. 25 and 26 depict a similar absorbent garment 1600 with first side portions 1166 that do not have the lower hip region. A front view of the absorbent garment 1600 in its fully assembled form as it would appear when worn is shown in FIG. 26. FIG. 25 illustrates the garment 1600 in an intermediate stage of assembly. At this stage, the garment 1600 is fully assembled except for the joinder at the side seams 1630. In FIG. 25, the garment 1600 is shown in a generally flattened condition as it appears during the intermediate stage of assembly.

The garment 1600 is shown with and will be described relative to first and second orthogonal axes 1602, 1604. The first axis 1602 may be referred to as the vertical axis 1602 and the second axis 1604 may be referred to as the lateral axis 1604. It will be understood by those having ordinary skill in the art that the vertical axis 1602 corresponds generally to a head-to-toe axis of a standing wearer and that the lateral axis 1604 corresponds generally to a side-to-side axis of a standing wearer.

FIG. 25 is a plan view of the body-facing side of the flattened garment 1600. In the flattened condition, the garment 1600 may have a generally symmetric hourglass shaped structure, but it may also have any other shape suitable for the given application. The flattened absorbent garment 1600 has a longitudinal axis 1603 extending from the portion of the garment 1600 that will conform to the front of the user to the portion of the garment that will conform to the rear of the user. The longitudinal axis 1603 of the flattened garment 1600 is orthogonal to the lateral axis 1604, which, as previously noted, extends from side to side relative to a wearer of the finished garment 1600.

The absorbent garment 1600 may have a main body 1140 and associated components that, with the exception of the backsheet 1644, are substantially similar to those of the absorbent garment 1100 of FIGS. 14-16. The absorbent garment 1600 includes side panels 1660 extending laterally outward from the main body lateral edges 1168. The side panels 1660 may be formed at least in part from the backsheet 1644. The side panels 1660 may be formed using only the backsheet 1644 or an elasticized composite side panel 1660 may be formed by bonding additional layers to the backsheet 1644.

Each side panel 1660 has a first side portion 1666 extending laterally outward from a main body lateral edge 1168 adjacent the first main body waist portion 1148 and a portion of the central main body portion 1152. The first side portion 1666 has a waist opening side panel edge 1670 and an outer lateral side panel edge 1672. Each side panel 1660 also has a second side portion 1674 extending laterally outward from the main body lateral edge 1168 adjacent the second main body waist portion 1150 and a portion of the central main body portion 1152. The second side portion 1674 also has a waist opening side panel edge 1678 and an outer lateral side panel edge 1680.

The first and second side portions 1666, 1674 are configured so that the body facing surface 1662 in the area adjacent the outer lateral side panel edge 1672 of the first side portion 1666 may be made to engage the body facing surface 1662 in the area adjacent the outer lateral side panel edge 1680 of the second side portion 1674 when the garment is folded to convert it from the configuration of FIG. 25 to the configuration of FIG. 26. With the body facing surface 1662 of the first and second side portions 1666, 1674 so-engaged, the first and second side portions 1666, 1674 may be joined by the formation of the side seam 1630 thereon. The first and second side seam portions 1666, 1674 are joined and the side seam 1630 is formed with the first and second side seam portions 1666, 1674 in a fully stretched condition.

The first and second side portions 1666, 1674 may be configured so that at least a portion of the outer lateral side panel edge 1672 of the first side portion 1666 is a mirror image of at least a portion of the outer lateral side panel edge 1680 of the second side portion 1674. Further, the first and second side portions 1666, 1674 may be configured so that when the body facing surface 1662 of the first and second side portions 1666, 1674 is engaged by folding the garment during manufacture, the waist opening side panel edges 1670, 1678 can be substantially aligned and at least a portion of the outer lateral side panel edges 1672, 1680 can be substantially aligned.

When the first and second side portions 1666, 1674 of both side panels 1660 are joined, the first and second main body waist portions 1148, 1150 combine with the side panels 1660 to form a waist encircling portion 1606, a waist opening edge 1631 and a waist opening 1632. Also, the central main body portion 1152 combines with the side panels 1660 to form a crotch region 1608, leg opening edges 1633 and two leg openings 1634. The partially curved side seam 1630 causes the completed side panels 1660 to adopt a pre-formed curvature that is not provided by previous garments having straight seams or seams formed only from straight segments.

In this embodiment, each side seam 1630 has a straight portion 1636 adjacent the leg opening 1634 on the same lateral side of the garment 1600 as the side seam 1630 and a curved portion 1638 adjacent the waist opening 1632. The curved portion 1638 of side seam 1630 may have any curvature that serves to enhance comfort and fit by better conformance with body curves of the wearer. For example, the geometry of the curved portion 1638 may include regular curve segments such as circular or elliptical arcs or can be formed from a plurality of similar or disparate regular curve segments. The curved portion 1638 may also be formed at least in part with a substantially free-form curve geometry.

Each side seam 1630 intersects the waist opening edge 1631 and one of the leg opening edges 1633. At the intersection with the waist opening edge 1631, a tangent to the centerline of the side seam 1630 defines a first seam angle θ_{S1} relative to the vertical. The straight seam portion 1636 defines a second seam angle with respect to the vertical at the intersection of the side seam 1630 with the leg opening edge 1633. In the embodiment illustrated in FIG. 11, the straight seam portion 1636 is substantially vertical, thus making the second seam angle equal to 0 degrees. It will be understood, however, that in other embodiments, the straight seam portion 1636 could be non-vertical, thus producing a non-zero second seam angle. The first seam angle θ_{S1} and the second seam angle may be designed specifically to accommodate different body types and sizes. For certain body types, the first and second seam angles may be equal. For other body types, the first and second seam angles may be significantly different. The first and second seam angles may each practicably be in a range of about 0 degrees to about 80 degrees. To accommodate the most typical body shapes, the first and second seam angles are each preferably in a range from about 10 degrees to about 50 degrees and most preferably in a range from about 25 degrees to about 35 degrees.

It will be understood that the final geometry of the waist opening side panel edges 1670, 1678 and the outer lateral side panel edges 1672, 1680 may be formed either before or after the side seam 1630 is formed to join the first and second side portions 1666, 1674. In either case, the lateral side panel edges 1672, 1680 may be formed so as to substantially match the geometry of the side seam 1630.

It will be understood by those having ordinary skill in the art that other embodiments of the present invention may include side panels formed using side seams that each have a first curved portion adjacent the waist opening, a second curved portion adjacent the leg opening and a straight portion between the first and second curved portions.

The various parts of the absorbent garments of the present invention can be attached to one another or associated with one another to form a structure that preferably maintains its shape during the useful life of the garment. As used herein, the terms “attached,” “joined,” “associated,” and similar terms encompass configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the respective parts of the garments to one another.

Due to the wide variety of backing and liner sheet construction and materials currently available, the invention is not intended to be limited to any specific materials or constructions of the components useful in the present invention. The backsheet 144 preferably is made from any suitable pliable liquid-impervious material known in the art. Typical backsheet materials include films of polyethylene, polypropylene, polyester, nylon, and polyvinyl chloride and blends of these materials. For example, the backsheet can be made of a polyethylene film having a thickness in the range of 0.02-0.04 mm. The backsheet 144 may be pigmented with, for example, titanium dioxide, to provide the garment with a pleasing color or to render the backsheet 144 opaque enough that exudates being contained by the garment are not visible from outside the garment. In addition, the backsheet 144 may be formed in such a manner that it is opaque, for example, by using various inert components in the polymeric film and then biaxially stretching the film. Other backsheet materials will be readily apparent to those skilled in the art. The backsheet 144 preferably has sufficient liquid imperviousness to prevent any leakage of fluids. The required level of liquid imperviousness may vary between different locations on the garment.

The backsheet 144 may further comprise separate regions having different properties. In a preferred embodiment, portions of the backsheet 144 are air-permeable to improve the breathability, and therefore comfort, of the garment. The different regions may be formed by making the backsheet 144 a composite or laminate of different sheet materials, chemical treatment, heat treatment, or other processes or methods known in the art. Some regions of the backsheet 144 may be fluid pervious. In certain embodiments of the invention, the backsheet 144 may be fluid impervious in the crotch region 108, but fluid pervious in portions of the first and second waist portions 110, 120.

The moisture-pervious topsheet 142 can be comprised of any suitable relatively liquid-pervious material known in the art that permits passage of liquid there through. Non-woven liner sheet materials are exemplary because such materials readily allow the passage of liquids to the underlying absorbent laminate core 146. Examples of suitable liner sheet materials include non-woven spunbond or carded webs of polypropylene, polyethylene, nylon, polyester and blends of these materials.

The backsheet 144 may be covered with a fibrous, nonwoven fabric such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded nonwoven web of synthetic fibers such as polypropylene, polyethylene or polyester fibers; a nonwoven web of cellulosic fibers, textile fibers such as rayon fibers, cotton and the like, or a blend of cellulosic and textile fibers; a spun-bonded nonwoven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the backsheet 144 may comprise three panels wherein a central poly backsheet panel is positioned closest to absorbent laminate core 146 while outboard non-woven breathable side backsheet panels are attached to the side edges of the central poly backsheet panel. Alternatively, the backsheet 144 may be formed from microporous poly coverstock for added breathability. The image of the invention can suitably be formed on any of these materials, including the fibrous, non-woven outer cover sheet.

The topsheet 142 may be formed of three separate portions or panels. Those skilled in the art will recognize, however, that topsheet 142 need not be made of three separate panels, and that it may be comprised of one unitary item. A first topsheet panel (not shown) may comprise a central topsheet panel formed from preferably a liquid-pervious material that is either hydrophobic or hydrophilic. The central topsheet panel may be made from any number of materials, including synthetic fibers (e.g., polypropylene or polyester fibers), natural fibers (e.g., wood or cellulose), apertured plastic films, reticulated foams and porous foams to name a few. One preferred material for a central topsheet panel or entire topsheet 142 is a cover stock of single ply non-woven material which may be made of carded fibers, either adhesively or thermally bonded, perforated plastic film, spunbonded fibers, or water entangled fibers, which generally weigh from 0.3-0.7 oz./sq. yd. and have appropriate and effective machine direction and cross-machine direction strength suitable for use as a baby diaper cover stock material. The central topsheet panel preferably extends from substantially the second waist portion 120 to the first waist portion 110, or a portion thereof.

A second and third topsheet panel (e.g., outer topsheet panels, not shown), in this alternative embodiment may be positioned laterally outside of the central topsheet panel. The outer topsheet panels preferably are substantially liquid-impervious and hydrophobic, preferably at least in the crotch area. The outer edges of the outer topsheet panels may substantially follow the corresponding outer perimeter of the backsheet 144. The material for the outer topsheet portions or panels preferably is polypropylene and can be woven, non-woven, spunbonded, carded or the like, depending on the application.

At the point of connection between the outer topsheet panels and the central topsheet panel, inner edges of the outer topsheet portions or panels may extend upwardly to form waste containment flaps (not shown). The waste containment flaps preferably are formed of the same material as the outer topsheet portions or panels, as in the embodiment shown. The waste containment flaps may be treated with a suitable surfactant to modify their hydrophobicity/hydrophilicity as desired, and they may be treated with skin wellness ingredients to reduce skin irritation. Alternatively, the waste containment flaps may be formed as separate elements and then attached to the body side liner.

The waste containment flaps preferably include a portion that folds over onto itself to form a small enclosure. At least one, and depending on the size of the enclosure sometimes more than one, elastic member may be secured in the enclosure in a stretched condition. As is known in the art, when the flap elastic attempts to assume the relaxed, unstretched condition, the waste containment flaps rise above the surface of the central topsheet portion or panel.

The topsheet 142 may be made of any suitable relatively liquid-pervious material currently known in the art or later discovered that permits passage of a liquid there through. Examples of suitable topsheet materials include nonwoven spun-bonded or carded webs of polypropylene, polyethylene, nylon, polyester and blends of these materials, perforated, apertured, or reticulated films, and the like. Nonwoven materials are exemplary because such materials readily allow the passage of liquids to the underlying absorbent core 146. The topsheet 142 preferably comprises a single-ply nonwoven material that may be made of carded fibers, either adhesively or thermally bonded, spunbonded fibers, or water entangled fibers, which generally weigh from 0.3-0.7 oz./sq. yd. and have appropriate and effective machine direction (longitudinal) and cross-machine (lateral) direction strength suitable for use as a topsheet material for the given application. The present invention is not intended to be limited to any particular material for the topsheet 142, and other topsheet materials will be readily apparent to those skilled in the art.

The topsheet 142 may further comprise several regions having different properties. In one embodiment of the present invention, the laterally distal portions of the topsheet 142, especially those used to make the outer topsheet panels preferably are substantially fluid impervious and hydrophobic, while the remainder of the topsheet 142 (e.g., central topsheet panel) is hydrophilic and fluid pervious. Different topsheet properties, such as fluid perviousness and hydrophobicity, may be imparted upon the topsheet 142 by treating the topsheet 142 with adhesives, surfactants, or other chemicals, using a composite of different materials, or by other means. The topsheet 142 may also be made from a laminate of overlaid sheets of material. The topsheet 142 also may be treated in specific areas like the crotch region, with skin wellness ingredients such as aloe, vitamin E, and the like.

As noted elsewhere herein, the topsheet 142 and backsheet 144 may be substantially coterminous, or they may have different shapes and sizes. The particular design of the topsheet 142 and backsheet 144 may be dictated by manufacturing considerations, cost considerations, and performance considerations. Preferably, the topsheet 142 is large enough to completely cover the absorbent laminate core 146, and the backsheet 144 is large enough to prevent leakage from the garment. The design of topsheet 142 and backsheet 144 is known in the art, and a skilled artisan will be able to produce an appropriate topsheet 142 and an appropriate backsheet 144 without undue experimentation.

The topsheet 142 and the backsheet 144 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as Cycloflex as sold by National Starch, a corporation headquartered in Bridgewater, N.J., is used to join the topsheet 142 to the backsheet 144. The particular joining method may be dictated by the types of materials selected for the topsheet 142 and backsheet 144.

As mentioned above, the absorbent garment preferably is provided with containment elastics 154 extending through the crotch region 108, adjacent the crotch edge 112. The absorbent garment of the invention also preferably is provided with waist elastic material optionally in the first and second waist portions, 110, 120, respectively, to enable and assist in stretching around the wearer. The waist elastics may be similar structures or different to impart similar or different elastic characteristics to the first and second waist portions 110, 120 of the garment. In general, the waist elastics may preferably comprise foam strips positioned at the first and second waist portions 110, 120, respectively. Such foam strips preferably are about ½ to about 1 ½ inches wide and about 3-6 inches long. The foam strips preferably are positioned between the topsheet 142 and the backsheet 144. Alternatively, a plurality of elastic strands may be employed as waist elastics rather than foam strips. The foam strips preferably are comprised of polyurethane, but can be any other suitable material that decreases waist band roll over, reduces leakage over the waist ends of the absorbent garment, and generally improve comfort and fit. The first and optional second waist foam strips preferably are stretched 50-150%, preferably 100% more than their unstretched dimension before being adhesively secured between the backsheet 144 and topsheet 142 or before being secured between two materials, preferably non-woven materials.

The lateral edges of the main body are preferably provided with an adjacent leg elastic containment system including containment elastics 154. In the preferred embodiment, three strands of elastic threads are positioned to extend adjacent to the leg openings 134 between the topsheet 142 and the backsheet 144. Any suitable elastomeric material exhibiting at least an elongation (defined herein as (L_{S}−L_{R})/L_{R} where L_{S} is the stretch length of an elastic element and L_{R} is retracted length, multiplied by 100 to obtain percent elongation) in the range of 5%-350%, preferably in the range of 200%-300%, can be employed for the containment elastics 154. The containment elastics 154 may be attached to the absorbent garment in any of several ways which are known in the art. For example, the containment elastics 154 may be ultrasonically bonded, heat/pressure sealed using a variety of bonding patterns, or glued to the garment. Various commercially available materials can be used for the containment elastics 154, such as natural rubber, butyl rubber or other synthetic rubber, urethane, elastomeric materials such as LYCRA (DuPont), GLOSPAN (Globe) or SYSTEM 7000 (Fulflex).

As stated previously, the invention has been described in connection with a diaper. The invention, however, is not intended to be limited to application only in diapers. Specifically, the fastening arrangements and absorbent cores of the described embodiments may be readily adapted for use in other absorbent garments besides diapers, including, but not limited to, training pants, feminine hygiene products and adult incontinence products. Indeed, given the enhanced thermal transmittance, the absorbent garments of the present invention are particularly suitable for use in adult incontinence products and feminine hygiene products.

The underlying structure beneath the topsheet 142 may include, depending on the diaper construction, various combinations of elements, but in each embodiment, it is contemplated that the absorbent garment will preferably include an absorbent core 146. For example, additional layers may be disposed between the topsheet 142 and absorbent core 146, and/or other additional layers may be disposed between these layers, or between absorbent core 146 and backsheet 144. The additional layer(s) may include a fluid transfer layer, a fluid handling layer, a storage layer, a wicking layer, a fluid distribution layer, and any other layer(s) known to those having ordinary skill in the art.

Although the absorbent core 146 depicted in FIG. 1 has a substantially rectangular cross-sectional and plan view shape, other shapes may be used, such as a “T” shape or an hourglass shape. The shape of the absorbent core 146 may be selected to provide the greatest absorbency with a reduced amount of material. The absorbent core may be associated with the topsheet 142, backsheet 144, or any other suitable part of the garment by any method known in the art, in order to fix the absorbent core 146 in place. In addition to the respective layers in the absorbent core 146, as will be described in greater detail hereinafter, the overall absorbent core 146 may be enclosed within a tissue wrapping, as disclosed in U.S. Pat. No. 6,068,620, the disclosure of which is incorporated by reference herein in its entirety. Skilled artisans are capable of designing and wrapping a suitable absorbent core 146 of the invention, using the guidelines provided herein.

Any suitable absorbent material may be used for absorbent core 146. Absorbent cores containing a mixture of fibrous material and super absorbent polymers (SAP) are well known in the art and described, for example, in U.S. Pat. No. 5,281,207, to Chmielewski, and U.S. Pat. No. 5,863,288, to Baker, the disclosures of each of which are herein incorporated by reference in their entirety and in a manner consistent with this disclosure. The fibrous material can be any fibrous material, preferably one that is capable of absorbing fluids and capable of retaining SAP particles within its matrix. Preferred fibrous materials may be selected from cellulose acetate fibers, rayon fibers, Courtauld's LYOCELL fibers, polyacrylonitrile fibers, surface-modified (hydrophilic) polyester fibers, surface-modified polyolefin/polyester bicomponent fibers, surface-modified polyester/polyester bicomponent fibers, cotton fibers, or blends thereof. In addition, rayon, Courtauld's LYOCELL, polyacrylonitrile, cotton fibers and cotton linters are alternatively preferred. The remaining fibers, surface-modified polyolefin/polyester bicomponent fibers, and surface-modified polyester/polyester bicomponent fibers are also believed to be effective fibrous materials for use in the invention.

Any SAP now known or later discovered may be used in absorbent core 146, so long as it is capable of absorbing liquids. Useful SAP materials are those that generally are water-insoluble but water-swellable polymeric substance capable of absorbing water in an amount that is at least ten times the weight of the substance in its dry form. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims and equivalents thereof.

What is claimed is:

1. An absorbent article comprising:
a main body having a first main body waist portion, a second main body waist portion and a central main body portion disposed between the first and second main body waist portions, the main body having orthogonal longitudinal and lateral axes; and
a pair of elastic side panels each connecting the first main body waist portion to the second main body waist portion, the main body and the side panels collectively defining a waist opening edge surrounding a waist opening and two leg opening edges, each leg opening edge surrounding a leg opening, each elastic side panel having a first side portion extending laterally outward from the first main body waist portion and terminating in a first outer lateral side panel edge, and a second side portion extending laterally outward from the second main body waist portion and terminating in a second outer lateral side panel edge, the first side portion being attached to the second side portion by a side seam, at least a portion of the side seam being curved when the first and second side portions are in a stretched condition.

2. An absorbent article according to claim 1 wherein the absorbent article comprises a backsheet and wherein the main body includes a first portion of the backsheet, a topsheet attached to the first portion of the backsheet and an absorbent core disposed intermediate the topsheet and the first portion of the backsheet, the elastic side panels being formed at least in part from a second portion of the backsheet.

3. An absorbent article according to claim 1 wherein the elastic side panels are formed as separate members each attached to the main body along at least a portion of a main body lateral edge.

4. An absorbent article according to claim 1 wherein the entire side seam is curved when the first and second side portions are in a stretched condition.

5. An absorbent article according to claim 1 wherein the at least a portion of the side seam is formed as a circular arc.

6. An absorbent article according to claim 1 wherein the at least a portion of the side seam comprises a plurality of curved sub-portions.

7. An absorbent article according to claim 6 wherein at least two of the plurality of curved sub-portions are formed with different geometric curve types.

8. An absorbent article according to claim 1 wherein a first portion of the side seam is curved and a second portion of the side seam is substantially straight when the first and second side portions are in a stretched condition.

9. An absorbent article according to claim 8 wherein the first portion of the side seam has a centerline that intersects the waist opening edge at a first intersection point, a tangent to the centerline defining a first seam angle with respect to the vertical axis.

10. An absorbent article according to claim 9 wherein the first seam angle is in a range from about 0 to about 80 degrees.

11. An absorbent article according to claim 9 wherein the first seam angle is in a range from about 10 degrees to about 50 degrees.

12. An absorbent article according to claim 9 wherein the first side seam angle is in a range from about 25 degrees to about 35 degrees.

13. An absorbent article according to claim 9 wherein the second portion of the side seam has a centerline that intersects one of the leg opening edges at a second intersection point, the centerline defining a second seam angle with respect to the vertical axis.

14. An absorbent article according to claim 13 wherein the second seam angle is in a range from about 0 to about 80 degrees.

15. An absorbent article according to claim 13 wherein the second seam angle is in a range from about 10 degrees to about 50 degrees.

16. An absorbent article according to claim 13 wherein the second side seam angle is in a range from about 25 degrees to about 35 degrees.

17. An absorbent article according to claim 8 wherein the first portion of the side seam has a centerline that intersects one of the leg opening edges at a first intersection point, a tangent to the centerline defining a first seam angle with respect to the vertical axis.

18. An absorbent article according to claim 17 wherein the first seam angle is in a range from about 0 to about 80 degrees.

19. An absorbent article according to claim 17 wherein the first seam angle is in a range from about 10 degrees to about 50 degrees.

20. An absorbent article according to claim 17 wherein the first side seam angle is in a range from about 25 degrees to about 35 degrees.

21. An absorbent article according to claim 17 wherein the second portion of the side seam has a centerline that intersects one of the leg opening edges at a second intersection point, the centerline defining a second seam angle with respect to the vertical axis.

22. An absorbent article according to claim 21 wherein the second seam angle is in a range from about 0 to about 80 degrees.

23. An absorbent article according to claim 21 wherein the second seam angle is in a range from about 10 degrees to about 50 degrees.

24. An absorbent article according to claim 21 wherein the second side seam angle is in a range from about 25 degrees to about 35 degrees.

25. An absorbent article according to claim 8 wherein the first portion of the side seam includes a first curved sub-portion having a first centerline that intersects the waist opening and a second curved sub-portion having a second centerline that intersects one of the leg openings, a first tangent to the first centerline defining a first seam angle with respect to the vertical axis and a second tangent to the second centerline defining a second seam angle with respect to the vertical axis.

26. An absorbent article according to claim 1 wherein the first side portion includes a lower hip region having a lateral hip region edge, the lateral hip region edge intersecting the first outer lateral side panel edge.

27. An absorbent article according to claim 26 wherein at least a portion of the first outer lateral side panel edge and at least a portion of the lateral hip region edge form a substantially continuous curve.

28. An absorbent article comprising:
a main body having a first main body waist portion, a second main body waist portion and a central main body portion disposed between the first and second main body waist portions, the main body having orthogonal vertical and lateral axes;
a pair of side panels, each side panel connecting the first main body waist portion to the second main body waist portion, the main body and the side panels collectively defining a waist opening edge and two leg opening edges, each side panel being formed from a first side portion extending laterally outward from the first main body waist portion and a second side portion extending laterally outward from the second main body waist portion; and
a pair of side seams, each side seam connecting the first side portion of one of the side panels to the second side portion of the same side panel, each side seam intersecting the waist opening edge at a first seam angle relative to the vertical axis and intersecting one of the leg opening edges at a second seam angle relative to the vertical axis when the first and second side portions of the associated side panel are in a fully stretched condition, at least a portion of each side seam being curved when the first and second side portions of the associated side panel are in a stretched condition.

29. An absorbent article according to claim 28 wherein at least one of the first and second side seam angles is in a range from about 10 degrees to about 80 degrees.

30. An absorbent article according to claim 28 wherein at least one of the first and second side seam angles is in a range from about 10 degrees to about 50 degrees.

31. An absorbent article according to claim 28 wherein at least one of the first and second side seam angles is in a range from about 25 degrees to about 35 degrees.

32. An absorbent article according to claim 28 wherein at least a portion of each side seam adjacent the waist opening edge is curved when the first and second side portions of the associated side panel are in a fully stretched condition.

33. An absorbent article according to claim 32 wherein at least a portion of each side seam adjacent the leg opening edge intersected by the side seam is substantially straight when the first and second side portions of the associated side panel are in a fully stretched condition.

34. An absorbent article according to claim 28 wherein at least a portion of the side seam adjacent the leg opening edge intersected by the side seam is curved when the first and second side portions of the associated side panel are in a fully stretched condition.

35. An absorbent article according to claim 34 wherein at least a portion of the side seam adjacent the waist opening edge is substantially straight when the first and second side portions of the associated side panel are in a fully stretched condition.

36. A method of forming an absorbent article comprising:
providing a backsheet having orthogonal longitudinal and lateral axes;
disposing an absorbent core on the backsheet;
attaching a topsheet to the backsheet with the absorbent core disposed intermediate the topsheet and the backsheet, the backsheet, the absorbent core and the topsheet collectively forming a main body having a first lateral edge and an opposing second lateral edge and having a first main body waist portion, a second main body waist portion and a central main body portion disposed between the first and second main body waist portions;
providing first and second side panels, each having a first side portion with a first body-facing surface, a first outward-facing surface and a first outer lateral side panel edge and having a second side portion with a second body facing surface, a second outward facing surface and a second outer lateral side panel edge;
attaching the first side panel to the main body along the first lateral edge so that the first side portion of the first side panel extends laterally outward from the first main body waist portion and the second side portion of the first side panel extends laterally outward from the second main body waist portion;
attaching the second side panel to the main body along the second lateral edge so that the first side portion of the second side panel extends laterally outward from the first main body waist portion and the second side portion of the second side panel extends laterally outward from the second main body waist portion;
folding the main body so that the first body-facing surface of the first side panel engages the second body facing surface of the first panel and the first body-facing surface of the second side panel engages the second body facing surface of the second panel;
forming a first side seam adjacent the first and second outer lateral edges of the first side panel to hold the first body facing surface of the first panel in permanent contact with the second body facing surface of the first panel, the first side seam being formed with the first side panel in a fully stretched condition and with at least a portion of the first side seam being curved; and
forming a second side seam adjacent the first and second outer lateral edges of the second side panel to hold the first body facing surface of the second panel in permanent contact with the second body facing surface of the second panel, the second side seam being formed with the second side panel in a stretched condition and with at least a portion of the second side seam being curved.

37. A method of forming an absorbent article comprising:
providing a generally planar backsheet having orthogonal longitudinal and lateral axes, a main backsheet portion and a first backsheet side portion extending laterally outward from a first lateral side of the main backsheet portion and a second backsheet side portion extending from a second lateral side of the main backsheet portion, the first lateral side being generally opposite the second lateral side;
disposing an absorbent core on the main backsheet portion;
attaching a topsheet to the backsheet with the absorbent core disposed intermediate the topsheet and the main backsheet portion, the main backsheet portion, the absorbent core and the topsheet collectively forming a main body having a first lateral edge and an opposing second lateral edge;
forming first and second side panels, the first side panel being formed at least in part from the first backsheet side portion and extending laterally outward from the first lateral edge and the second side panel being formed at least in part from the second backsheet side portion and extending laterally outward from the second lateral edge, each of the first and second side panels having a first side portion with a first body-facing surface, a first outward-facing surface and a first outer lateral side panel edge and having a second side portion with a second body facing surface, a second outward facing surface and a second outer lateral side panel edge;
folding the main body so that the first body-facing surface of the first side panel engages the second body facing surface of the first panel and the first body-facing surface of the second side panel engages the second body facing surface of the second panel;
forming a first side seam adjacent the first and second outer lateral edges of the first side panel to hold the first body facing surface of the first panel in permanent contact with the second body facing surface of the first panel, the first side seam being formed with the first side panel in a fully stretched condition and with at least a portion of the first side seam being curved; and
forming a second side seam adjacent the first and second outer lateral edges of the second side panel to hold the first body facing surface of the second panel in permanent contact with the second body facing surface of the second panel, the second side seam being formed with the second side panel in a fully stretched condition and with at least a portion of the second side seam being curved.
